(19) United States
(12) Patent Application Publication
(10) Pub. No.: US 2024/0342477 A1
(43) Pub. Date: Oct. 17, 2024

(54) PHRENIC NERVE PACING DEVICE

(71) Applicants: Texas Medical Center, Houston, TX (US); Texas Heart Institute, Houston, TX (US)

(72) Inventors: Lance Black, Pearland, TX (US); Skylar Buchan, Houston, TX (US); Elizabeth Jaworski, Houston, TX (US); Mathews M. John, Houston, TX (US); Allison Post, Houston, TX (US); Mehdi Razavi, Houston, TX (US)

(21) Appl. No.: 18/294,893

(22) PCT Filed: Aug. 2, 2022
(86) PCT No.: PCT/US2022/039188

§ 371 Date: Feb. 2, 2024

Related U.S. Application Data
(60) Provisional application No. 63/228,939, filed on Aug. 3, 2021.

(51) Int. Cl. A61N 1/36; A61N 1/05

(52) U.S. Cl. CPC A61N 1/3611; A61N 1/0519

(57) ABSTRACT

Disclosed herein, in some aspects, are endotracheal stimulation platforms, systems, and methods of their use for pacing the phrenic nerves, right and/or left phrenic nerves, of a subject. In some embodiments, one or more pairs of electrodes are provided through an ET tube to contact a portion of a tracheal wall, or trachea, so as to enable phrenic nerve pacing. In some cases, the one or more pairs of electrodes are configured to send electrical pulses that stimulate the phrenic nerve, thereby helping promote contraction and relaxation of the diaphragm muscle.

### CROSS-REFERENCE TO RELATED APPLICATIONS

This application claims the benefit of and priority to U.S. Provisional Application No. 63/228,939, filed Aug. 3, 2021, the entire contents of which are incorporated by reference herein.

### BACKGROUND

Phrenic nerve stimulation is a technique used to pace the diaphragm of patients with central nervous system etiologies of respiratory insufficiency.

### SUMMARY

In a first aspect, the invention provides an endotracheal stimulation system including: a catheter wherein at least a distal portion of the catheter includes a pair of electrodes including a proximal looped electrode and a distal looped electrode, wherein the proximal looped electrode and the distal looped electrode are in electrical communication with a proximal portion of the system for connection to a control unit; and a guide sheath having a lumen surrounding at least a portion of the catheter and sized to fit within an endotracheal tube, wherein the catheter is axially translatable with respect to the endotracheal tube; wherein the endotracheal stimulation system has: a constrained configuration when at least a portion of the proximal looped electrode and the distal looped electrode are within the guide sheath; and an unconstrained configuration when at least a portion of the proximal looped electrode and the distal looped electrode extends out of the guide sheath such that the pair of electrodes contact the trachea of a subject.

In some embodiments, the proximal looped electrode and the distal looped electrode are bipolar. In some embodiments, the proximal looped electrode and the distal looped electrode are monopolar. In some embodiments, the proximal looped electrode is coated with a proximal insulation, wherein the distal looped electrode is coated with a distal insulation, or both. In some embodiments, the proximal looped electrode and the distal looped electrode are electrically isolated in the constrained configuration, the unconstrained configuration, or both. In some embodiments, a proximal point of the proximal looped electrode is proximal to a proximal point of the distal looped electrode by an electrode offset in the constrained configuration, the unconstrained configuration, or both. In some embodiments, the electrode offset is measured as a distance between a distal point of the proximal looped electrode and a distal point of the distal looped electrode In some embodiments, the electrode offset is about 0.25 cm to about 16 cm.

In another aspect, the invention provides an endotracheal stimulation system including: an catheter wherein at least a distal portion of the catheter includes a pair of electrodes, wherein the pair of electrodes are in electrical communication with a proximal portion of the system for connection to a control unit, and wherein the distal portion of the catheter is biased to have a shape that is non-straight when in the unconstrained configuration; and a guide sheath having a lumen surrounding at least a portion of the catheter and sized to fit within an endotracheal tube, wherein the catheter is axially translatable with respect to the guide sheath; wherein the endotracheal stimulation system has: a constrained configuration when the distal portion of the catheter is within the guide sheath, and wherein an axis of the catheter and an axis of the guide sheath are axially aligned; and an unconstrained configuration when the distal portion of the catheter extends out of the guide sheath, wherein at least a portion of the axis of the catheter and the axis of the guide sheath are askew, such that the pair of electrodes contact a trachea of a subject.

In some embodiments, the pair of electrodes are bipolar. In some embodiments, the pair of electrodes are monopolar. In some embodiments, the distal portion of the catheter includes two or more pairs of electrodes. In some embodiments, the two or more pairs of electrodes are distributed along the length of the catheter. In some embodiments, each of the two or more pairs of electrodes are separated by an offset distance of about 0.25 cm to about 16 cm. In some embodiments, the catheter further includes a lumen. In some embodiments, the catheter is flexible, pliable, or both. In some embodiments, the catheter has a non-straight shape in the unconstrained configuration with a diameter of about 1 mm to about 35 mm. In some embodiments, the catheter has a non-straight shape in the unconstrained configuration with a minimum, average, or maximum arc angle of about 90 degrees to about 360 degrees.

In another aspect, the invention provides an endotracheal stimulation system including: a catheter at the distal end of the system configured to wrap around at least a portion of an endotracheal tube, wherein the catheter includes a pair of electrodes on an outer catheter surface, and wherein the pair of electrodes are in electrical communication with a proximal portion of the system for connection to a control unit.

In some embodiments, the pair of electrodes are bipolar. In some embodiments, the pair of electrodes are monopolar. In some embodiments, the catheter includes two or more pairs of electrodes. In some embodiments, the two or more pairs of electrodes are evenly distributed along the length of the catheter.

In another aspect, the invention provides a method of pacing a subject, the method including: inserting the first system into a tracheal tube of a subject; contacting a wall of the trachea with the pair of electrodes by advancing the catheter out of the guide sheath to convert the endotracheal stimulation system from the constrained configuration to the unconstrained configuration; and providing electrical power to the pair of electrodes in a pattern to pace the phrenic nerve of the subject. In some embodiments, the method further includes flexing the catheter, deforming the catheter, or both.

In another aspect, the invention provides a method of pacing a subject, the method including: inserting the second system into a tracheal tube of a subject; contacting a wall of the trachea with the pair of electrodes by advancing the catheter out of the guide sheath to convert the endotracheal stimulation system from the constrained configuration to the unconstrained configuration; and providing electrical power to the pair of electrodes in a pattern to pace the phrenic nerve of the subject. In some embodiments, the method further includes flexing the catheter, deforming the catheter, or both.

In another aspect, the invention provides a method of pacing a subject, the method including: inserting the third system into the trachea of a subject; contacting a wall of the trachea with the pair of electrodes; and providing electrical power to the pair of electrodes in a pattern to pace the phrenic nerve of the subject. In some embodiments, the method further includes flexing the catheter, deforming the catheter, or both.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an illustration showing the position of the phrenic nerves of a subject. The illustration shows the oral cavity 001, the trachea 010, the left phrenic nerve 020, the right phrenic nerve 030, the spine 040, the esophagus 050, the bronchus 060, the carina 065, the left lung 070, the right lung 080, and the diaphragm 090. As shown, the left 020 and right 030 phrenic nerves run from the neck to branch in parallel from both sides of the spine at the C3, C4, and C5 vertebrae cervical and terminate at the diaphragm 090.

FIG. 2 is an illustration showing an exemplary endotracheal stimulation platform 1000. The system includes an endotracheal tube 200 configured to be inserted into the trachea 010 of a subject, an endotracheal tube connector 300 connecting the endotracheal tube 200 to the mechanical ventilator 400 by way of the ventilator tubing 410. The endotracheal tube connector 300 is also shown connecting the endotracheal tube 200 to the control unit 500 via two data cables 320. A sensor 310 is shown between the ventilator tubing 410 and the endotracheal tube connector 300. The first endotracheal system 100 is shown inside the trachea 010 of the subject in the exemplary form of two looped electrodes. Anatomical landmarks are also depicted including the larynx 005, the esophagus 050, and the carina 065.

FIG. 3 is an illustration showing the catheter 120 being introduced into the endotracheal tube connector 300 through the endotracheal tube connector port 340. The catheter 120 is shown traversing the guide sheath 110 through the guide sheath lumen. The locking mechanism 330 secures the data cable 320 to the endotracheal tube connector port 340. The ventilator tubing 410 connects with the proximal end of the endotracheal tube connector 300. The distal end of the endotracheal tube connector 300 is configured to connect with the proximal end of the endotracheal tube 200.

FIG. 4A is a side-view illustration showing an exemplary first endotracheal stimulation system 100 in a constrained configuration. The catheter 120 and the guide sheath 110 are also shown.

FIG. 4B is a side-view illustration showing an exemplary first endotracheal stimulation system 100 between the constrained configuration and an unconstrained configuration. The distal looped electrode 130 is shown in an unconstrained configuration while the proximal looped electrode 140 is shown in a constrained configuration. The distal catheter insulation 131 and the proximal catheter insulation 141, and the catheter 120 are shown near the opening at the distal end of the guide sheath 110.

FIG. 4C is a side-view illustration showing an exemplary first endotracheal stimulation system 100 in the unconstrained configuration. The distal looped electrode 130 and the proximal looped electrode 140 are shown extending beyond the opening at the distal end of the guide sheath 110 and unconstrained by the guide sheath 110. The distal catheter insulation 131, the proximal catheter insulation 141, the catheter 120 are also shown.

FIG. 5A is a top-view illustration showing an exemplary first endotracheal stimulation system 100 in the unconstrained configuration and where the distal looped electrode 130 and the proximal looped electrode 140 are in contact with the tracheal wall 015 of a subject. The distal looped electrode 130 and the proximal looped electrode 140 are physically separated from each other by an electrode offset 133. The distal catheter insulation 131, the proximal catheter insulation 141, the catheter 120, the guide sheath 110 are shown extending through the opening at the distal end of the endotracheal tube 202. The endotracheal tube 200 is also shown having a Murphy eye 220 opening positioned near the opening 204 at the distal end of the endotracheal tube. The endotracheal tube cuff 210 is also shown in contact with the tracheal wall 015. The endotracheal tube lumen 205 is also shown.

FIG. 5B is an illustration showing a perspective view of an exemplary first endotracheal stimulation system 100 in the unconstrained configuration. The distal looped electrode 130 and the proximal looped electrode 140 are physically separated from each other by an electrode offset 133. The distal catheter insulation 131 and the proximal catheter insulation 141 are shown extending through the opening at the distal end of the catheter 120.

FIG. 6A is an illustration showing a side-view of an exemplary second endotracheal stimulation system 600 in the constrained configuration. The catheter 620 and the guide sheath 610 are shown extending through the distal opening 203 of the endotracheal tube 200. The endotracheal tube lumen 205 is also shown.

FIG. 6B is an illustration showing a side view of an exemplary second endotracheal stimulation system 600 in the unconstrained configuration. The catheter 620 is shown having multiple pairs of electrodes. Each pair of electrodes 630 shown includes a distal electrode 631 and a proximal electrode 632. The guide sheath 610 is shown extending through the opening at the distal end of the endotracheal tube 200.

FIG. 6C is an illustration showing a top view of an exemplary distal region 625 of a catheter 620 and four pairs of electrodes disposed along the length of the catheter 620 in the unconstrained configuration. The catheter 620 is shaped as a loop having a catheter outer diameter 660. The distance from an outer edge of a first pair of electrodes 630 to the outer edge of a second pair of electrodes is shown as the electrode outer diameter 670. A distance along the length of the catheter 620 and between a distal electrode 631 and a proximal electrode 632 is shown as the electrode offset 633. A distance along the length of the catheter 620 that separates a first pair of electrodes from a second pair of electrodes is shown as the electrode pair offset 635. The electrode length along the length of the catheter 640, the electrode width 650, an electrode outer edge 651, an electrode inner edge 652, and the electrode outer diameter 670 are also shown.

FIG. 7 is an illustration showing a side-view of a distal portion of an exemplary third endotracheal stimulation system 700. The endotracheal tube 200 having an endotracheal tube cuff 210 at the distal end of the endotracheal tube 202, and an opening 204 at the distal end of the endotracheal tube is shown. A spiral-shaped catheter 720 is shown having a plurality of electrode pairs 730 and disposed along the length of the endotracheal tube 200. The spiral-shaped catheter 720 wraps around the endotracheal tube 200 from the proximal end of the endotracheal tube 201 (not shown) to the distal end of the endotracheal tube 202. Each pair of electrodes 730 contains a distal electrode 731 and a proximal electrode 732. The distal electrode 731 and the proximal electrode 732 are separated by an electrode offset 733 distance along the length of the spiral-shaped catheter 720. A pilot balloon line 215 is also shown.

FIG. 8A is an illustration showing a side view of the distal portion of a fourth endotracheal stimulation system 700. The endotracheal stimulation system shown has a collar-shaped catheter. The pair of electrodes 730 are configured to wrap around the endotracheal tube 200. Each pair of electrodes 730 includes a distal electrode 731, a proximal electrode 732, and a distance separating the distal electrode 731 from the proximal electrode called the electrode offset 730. Endotracheal stimulation system 700 is also shown having a gap 751, a prong 722, and an endotracheal tube 200 having a cuff 210 and a pilot balloon line 215.

FIG. 8B is an illustration showing a first collar-shaped catheter 750 of an exemplary third endotracheal stimulation system 700. A distal electrode 731, a proximal electrode 732, and a middle electrode 734 are shown at the distal end of the catheter.

FIG. 8C is an illustration showing a second collar-shaped catheter 751 of an exemplary third endotracheal stimulation system 700. A distal electrode 731 and a proximal electrode 732 are shown at the distal end of the catheter 720. The prong 722 and the gap 740 are also shown. The catheter is shown having an electrode portion 735.

FIG. 8D is an illustration showing a third collar-shaped catheter 752 of an exemplary third endotracheal stimulation system 700. Multiple distal electrodes 731 and proximal electrodes 732 are shown at the distal end of the catheter. The prong 722 and the gap 740 are also shown.

FIG. 9A is an illustration showing an exemplary fourth endotracheal stimulation system 800 having a catheter cuff 850, being fitted onto an endotracheal tube 200, in the constrained configuration. An endotracheal cuff 210 and a pilot balloon line 215 are also shown.

FIG. 9B is an illustration showing an exemplary fourth endotracheal stimulation system 800 having a catheter cuff 850 in the unconstrained configuration. The catheter cuff 850 is shown overlapping with the endotracheal cuff (not shown). Multiple cuff electrodes 830 are arranged circumferentially around the endotracheal tube 200 and are separated from each other by a cuff electrode offset 833.

### DETAILED DESCRIPTION

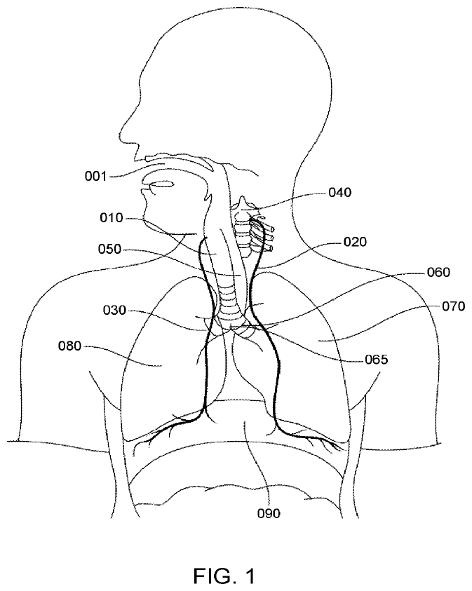

Provided herein are endotracheal stimulation platforms, systems, and methods of their use for pacing the phrenic nerves, right and/or left phrenic nerves (FIG. 1), of a subject. In some embodiments, one or more pairs of electrodes are provided through an ET tube to contact a portion of a tracheal wall, or trachea, so as to enable phrenic nerve pacing. For example, in some cases, the one or more pairs of electrodes are configured to send electrical pulses that stimulate the phrenic nerve, thereby helping promote contraction and relaxation of the diaphragm muscle. Such contraction and relaxation of the diaphragm muscles help promote the inhalation and exhalation phases of breathing.

### Endotracheal Stimulation Platforms

In general, the endotracheal stimulation (ETS) platforms of the invention may include one or more endotracheal stimulation systems, one or more control units, and/or one or more ventilators functionally connected therewith. In some embodiments, the ETS platform 1000 may include a one or more (e.g., 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) ETS systems. In some embodiments, the ETS systems are identically configured. In some embodiments, each ETS system is configured differently to the other ETS systems in an ETS platform. In some embodiments, the ETS platform 1000 includes one or more ETS systems of any combination of configurations.

The ETS platform 1000 may, in some embodiments, include one or more control units 500. The control unit 500 may be a hardware system, a software system, or a combined hardware and software system. In some embodiments the control unit 500 is one or more computing devices. In some embodiments, the control unit 500 includes one or more data input and data output channels for unidirectional and/or multidirectional (e.g., bidirectional, between three or more devices, between four or more devices, between five or more devices, between six or more devices, between seven or more devices, between eight or more devices, between nine or more devices, or between ten or more devices) data transfer between the control unit 500 and any one or more of the ETS platform 1000 components (e.g., one or more ETS systems and/or ventilator systems). In some embodiments, the control unit 500 determines that the subject is in an inspiratory or expiratory cycle based on the data measured by the sensor 310, and one or more patient monitoring devices as further described below.

In some embodiments, the control unit 500 provides electricity to at least a portion of the one or more pairs of electrodes as described herein for any endotracheal stimulation system described herein. In some embodiments, the catheter 120 comprises two or more pairs of electrodes, wherein the control unit 500 provides electricity to at least a portion of the two or more pairs of electrodes 150 based on a position of the two or more pairs of electrodes in the trachea 010 of the subject. In some embodiments, the control unit 500 controls the electric current provided to the one or more pair of electrodes. In some embodiments, the control unit 500 is programmable to output electricity to the one or more pairs of electrodes 150 for controlled pacing. In some embodiments, controlled pacing includes one or more controlled pacing parameters such as intensity, frequency, and duration. In some embodiments, the intensity is based on current, voltage, or both. In some embodiments, the control unit 500 is programmable to output electricity to the one or more pair of electrodes 150 based on data received by the sensor 310. In some embodiments, the control unit 500 employs a machine learning algorithm and/or artificial intelligence to optimize the subject's airflow. In some embodiments, the control unit 500 is further programmed to stop pacing and/or ventilation if on one or more measured subject parameters (e.g., blood pressure, blood oxygen content) is outside a set threshold.

In some embodiments, the ETS platform 1000 includes one or more ventilators. In some embodiments, the ventilator is any conventional device, apparatus, or system for delivering breathing gas to a patient, e.g., a ventilator, a respirator, a CPAP device, or a BiPAP device, among others. In some embodiments, the ventilator is capable of providing a continuous mandatory ventilation (CMV) output, that is, an output with parameters set by an operator without any feedback from the subject. In general, the ventilator provides at its output a cyclical supply of breathing gas at regulated pressure and at a frequency appropriate for ventilating the subject. The invention could be used with other sources of breathing gas at a regulated pressure suitable for supply to a subject, such as, for example any device capable of providing a CPAP output. One type of ventilator does not require any electrical supply and is powered exclusively by gas pressure, such as from an oxygen cylinder. Alternatively, the ventilator could be a simple electrical ventilator with a compressor that charges a gas pressure vessel.

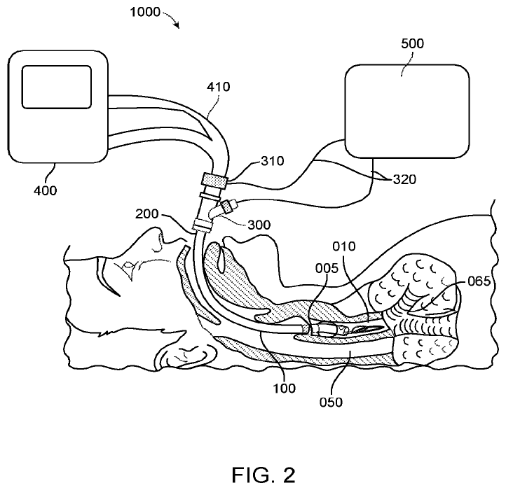

In some embodiments, the ETS platform 1000 includes one or more monitoring devices. In some embodiments, one or more monitoring devices are functionally connected to other components of the ETS platform 1000 (e.g., a ventilator and/or the control unit). In some embodiments, the monitoring devices are capable of collecting and displaying (either on its own display or another connected device having a display) information concerning one or more physiological parameters of the subject. In some embodiments, the physiological parameters that may be monitored include a subject's vital signs such as body temperature, pulse rate (or heart rate), blood pressure, oxygen saturation, respiratory rate, heart rate variability, pulse pressure strength, concentration of substances in the blood and/or tissue, similar parameters indicative of the human body vitality, or any combination thereof. In some embodiments, the endotracheal stimulation platform 1000 further comprises one or more subject sensors. In some embodiments, the one or more subject sensors comprises a skin contact electrode, an accelerometer, a thermometer, a pressure sensor, an air sensor 310 or any combination thereof. In some embodiments, the one or more subject sensors are in wired communication (e.g., as one or more data cables 320), as shown in FIG. 2, and/or in wireless communication with the control unit 500.

### Endotracheal Stimulation Systems

The endotracheal stimulation (ETS) systems herein described generally include a guide sheath, a catheter, an endotracheal (ET) tube, an ET tube connector, a sensor, and one or more pairs of electrodes. Exemplary embodiments of the ETS systems of the invention are further described in detail below and in the drawings (FIGS. 2-9B).

The ETS systems of the invention, generally, are configured to be in electrical communication with the control unit 500 of an ETS platform 1000 (FIG. 2). In some embodiments, the electrical communication is wired, wireless, or both. In some embodiments, one or more wires run through one or more data cables and are configured to connect to the one or more electrodes of any one or more embodiments herein described. In some embodiments, the ETS system includes a locking mechanism 330 to ensure that electrical communication is maintained throughout its operation. In some embodiments, the locking mechanism may be any reversible locking mechanism known in the art (e.g., clutch lock, cam lock, shock lock, spring button lock, or swaging lock). In some embodiments, the locking mechanism secures the ETS system to the endotracheal tube connector port 340. In some embodiments, the locking mechanism 330 is used to ensure the ETS system is coupled in place with the ET tube connector 300.

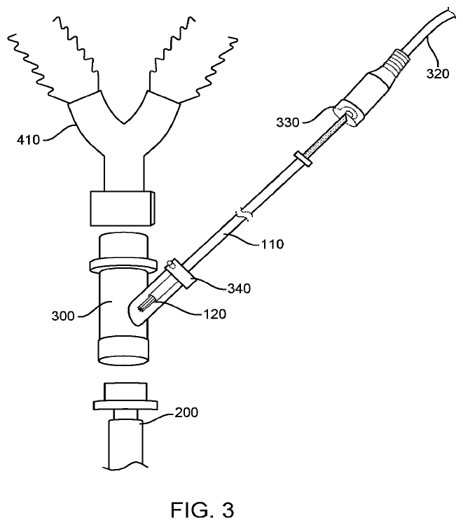

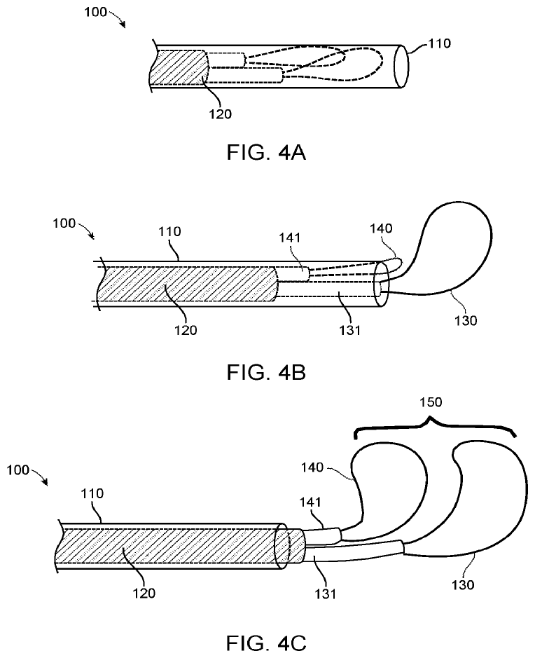

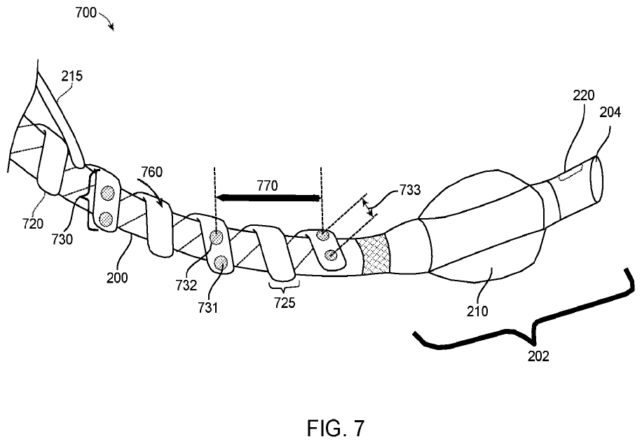

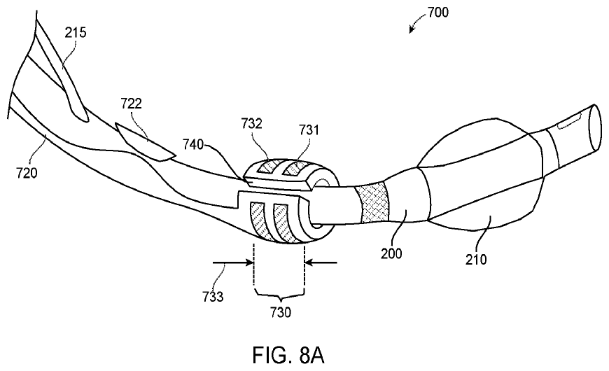

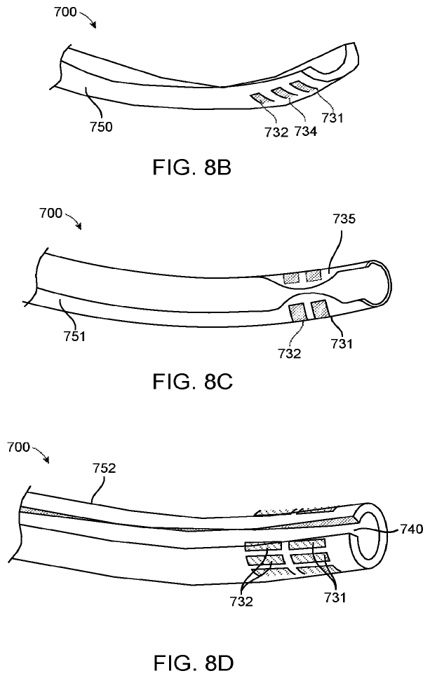

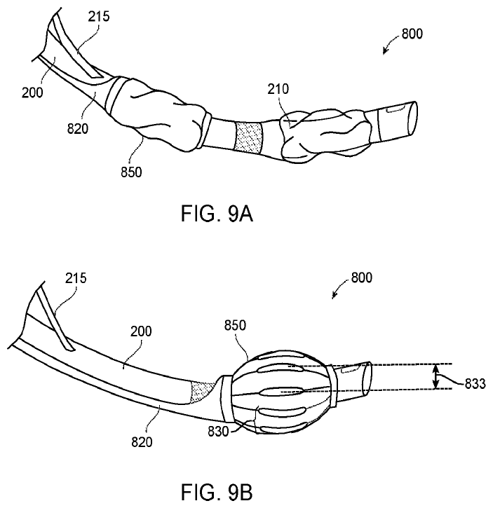

In some embodiments, the one or more pairs of electrodes of the ETS system are configured to be positioned into the site of endotracheal stimulation through the lumen of an ET tube 205 (FIG. 2, FIG. 3, FIGS. 4A-4C, FIGS. 5A-5B, and FIGS. 6A-6C). In some embodiments, the one or more pairs of electrodes are configured to be positioned into the site of endotracheal stimulation around the ET tube 200 (FIG. 7, FIGS. 8A-8D). In some embodiments, the one or more pairs of electrodes are configured to be positioned into the site of endotracheal stimulation around an ET tube cuff 210 (FIGS. 9A-9B).

In some embodiments, the ETS systems include one or more pair of electrodes wherein each pair of electrodes includes at least one distal electrode and at least one proximal electrode. In some embodiments, the distal electrode is positioned distal to the proximal electrode and closer to the distal end of the ETS system. The proximal-to-distal axis is generally used herein with relation to the longitudinal axis of the ET tube. For example, the distal electrode is at least partially positioned closer to the distal end of the ET tube the proximal electrode. In some embodiments, a first pair of electrodes may be positioned distal to a second pair of electrodes. In some embodiments, a plurality of pairs of electrodes may be disposed along the longitudinal axis of the ET tube in a proximal-to-distal configuration. In some embodiments, the guide sheath, the catheter, and/or the one or more pair of electrodes are translatable along the longitudinal axis, the longitudinal axis extending from the proximal end of the ET tube 200 to the distal end of the ET tube 200.

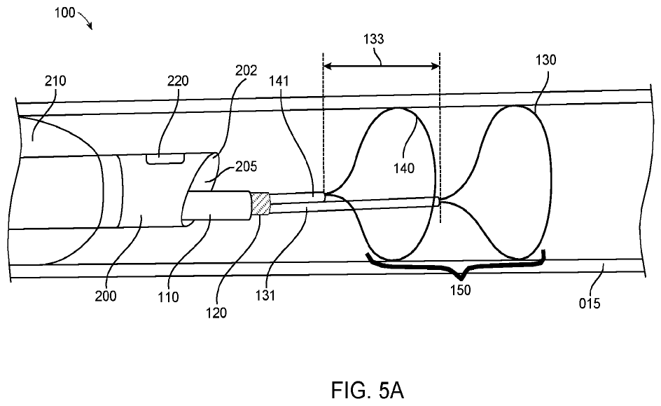

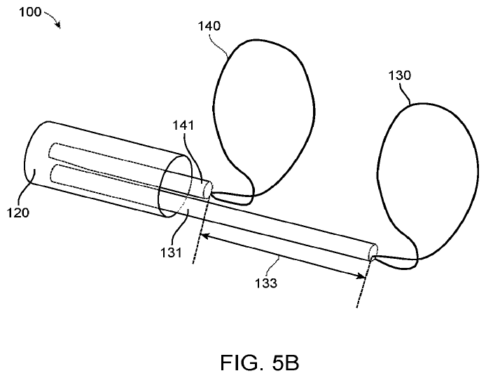

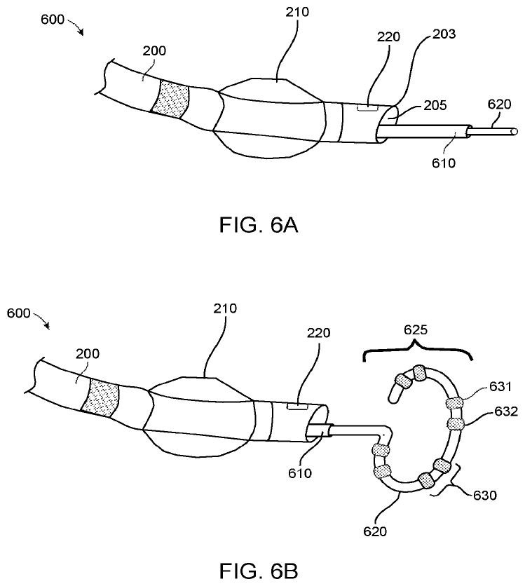

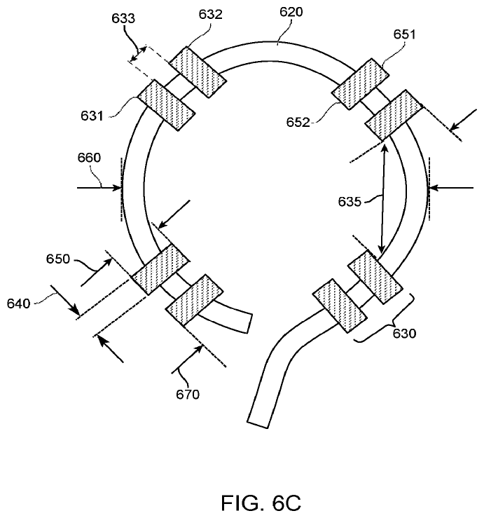

In some embodiments, the catheter 120 of any of the ETS systems herein described may include a catheter lumen. In some embodiments, the catheter is shaped as a cylindrical rod having a lumen through which one or more pairs of electrodes may be inserted and delivered to an endotracheal stimulation site as shown in FIG. 2, FIGS. 4A-4C, and FIGS. 5A-5B. In some embodiments, the catheter 620 is a support configured to underpin one or more pairs of electrodes thereon as shown in FIGS. 6A-6C, FIG. 7, and FIGS. 8A-8D. In some embodiments, the catheter is configured to be translatable along the length of an ET tube 200 through an ET tube lumen 205 (FIG. 5A). In some embodiments, the catheter is shaped to concentrically surround the ET tube (e.g., in a collar configuration as shown in FIGS. 8A-8D and FIGS. 9A-9B) and configured to be translatable along the length of an ET tube 200.

In some embodiments, the ETS systems include an ET tube 200 as seen in FIG. 7. In some embodiments, the ET tube includes an ET tube cuff 210, a Murphy's eye 220, an ET tube proximal end 201, an ET tube distal end 202, an opening at the ET tube proximal end 203, an opening 204 at the ET tube distal end, and an ET tube lumen 205.

### First Endotracheal Stimulation System

In a first aspect, the ETS platform 1000 includes a first ETS system 100 (FIGS. 2-5B). The first ETS system may, in some embodiments, include an ET tube 200, an ET tube connector 300, a guide sheath 110, a catheter 120, a sensor 310, and one or more pairs of electrodes 150 (FIG. 4A-4C). FIG. 3 shows the catheter 120 being introduced into the endotracheal tube connector 300 through the endotracheal tube connector port 340. The catheter 120 is shown traversing the guide sheath 110 through the guide sheath lumen. The locking mechanism 330 secures the data cable 320 to the endotracheal tube connector port 340. The ventilator tubing 410 connects with the proximal end of the endotracheal tube connector 300. The distal end of the endotracheal tube connector 300 is configured to connect with the proximal end of the endotracheal tube 200. In some embodiments, one or more components of the first ETS system are configured to be located within an ET tube 200, wherein the ET tube 200 is configured to be inserted into the trachea of a subject. In some embodiments, the ETS system does not comprise the catheter 120, wherein the pairs of electrodes are supported by the ET tube.

### Electrodes

FIGS. 4A-5B depict a distal portion of the first ETS system wherein a pair of electrodes 150 includes distal electrode 130 and proximal electrode 140. In some embodiments, the distal end of each electrode is shaped in a loop (FIGS. 4A-5B). In some embodiments, the looped electrodes 130 and 140 are located at the distal end of the catheter 120. In some embodiments, the ETS system 100 has a constrained configuration (FIG. 4A) wherein the electrode 130, 140 is disposed within the guide sheath 110. In some embodiments, the ETS system has an unconstrained configuration (FIG. 4C) wherein the electrode 130, 140 extends from a distal end of the guide sheath 110. In some embodiments, the ability of the proximal electrode 140 and the distal electrode 130 to convert from the constrained configuration to the unconstrained configuration enables catheters 120 of reduced sizes to be used with the ETS system, which impart less stress on the trachea and/or other internal organs. In some embodiments, in the constrained configuration at least a portion of the proximal electrode 140 and the distal electrode 130 are within the lumen of the guide sheath 110. In some embodiments, in the unconstrained configuration at least a portion of the proximal electrode 140 and the distal electrode 130 extends out of the guide sheath 110. In some embodiments, the proximal electrode 140) and the distal electrode 130 translate independently within the guide sheath 110. In some embodiments, in the constrained configuration the proximal electrode 140 and the distal electrode 130 do not touch the ET tube 200 or the trachea of the subject. In some embodiments, in the constrained configuration the proximal electrode 140 and the distal electrode 130 are fully within the ET tube 200. In some embodiments, the proximal electrode 140 and the distal electrode 130 are electrically isolated in the constrained configuration, the unconstrained configuration, or both.

In some embodiments, the ETS system includes a distal electrode 130 (FIGS. 4B-5B) and a proximal electrode 140 (FIGS. 4B-5B), wherein the distal electrode 130 is at least partially enveloped by the distal insulator 131 and the proximal electrode 140 is at least partially enveloped by the proximal insulator 141. In general, the pair of electrodes 150 is translatable along a longitudinal axis, the longitudinal axis extending from the proximal end of the ET tube 200 to the distal end of the ET tube 200. In some embodiments, the pair of electrodes 150 is translatable along the length of the ET tube 200.

In some embodiments, the electrodes 130) and/or 140 have a length of from about 1 cm to about 100 cm (e.g., about 1 cm, about 2 cm, about 3 cm, about 4 cm, about 5 cm, about 6 cm, about 7 cm, about 8 cm, about 9) cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the electrodes 130 and/or 140 have a length of from 10 cm to 100 cm (e.g., from 10 cm to 90 cm, from 10 cm to 80 cm, from 10 cm to 70 cm, from 10 cm to 60 cm, from 10 cm to 50 cm, from 10 cm to 40 cm, from 10 cm to 30 cm, from 20 cm to 90 cm, from 30 cm to 80 cm, from 40 cm to 70 cm, or from 50 cm to 60 cm). In some embodiments, the electrodes 130 and/or 140 have a length of at least about 1 cm, 5 cm, 10 cm, 25 cm, or 50 cm. In some embodiments, the electrodes 130 and/or 140 have a length of at most about 500 cm, 250 cm, 100 cm, 75 cm, 50 cm, 25 cm, or 10 cm.

In some embodiments, the loop of the electrode 130 and/or 140 has a diameter of from about 1 mm to about 35 mm (e.g., about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, or about 35 mm). In some embodiments, the loop has a diameter of from 1 mm to 35 mm (e.g., from 2 mm to 35 mm, from 5 mm to 35 mm, from 10 mm to 30 mm, from 15 mm to 25 mm, from 20 mm to 30 mm, or from 25 mm to 35 mm). In some embodiments, the loop of the electrode 130 and/or 140 has a diameter of at least about 1 mm, 5 mm, 10 mm, 15 mm, or 20 mm. In some embodiments, the loop of the electrode 130 and/or 14 has a diameter of at most about 35 mm, 30 mm, or 25 mm.

In some embodiments, the loop of the electrode 130 and/or 140 has a circumferential length (e.g., from one end of the loop to the other end of the loop) of from about 3 mm to about 110 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40) mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, or about 110 mm). In some embodiments, the loop of the electrode 130 and/or 140 has a circumferential length of from 3 mm to 110 mm (e.g., from 5 mm to 100 mm, from 10 mm to 90 mm, from 20 mm to 80 mm, from 30 mm to 70 mm, from 40 mm to 60 mm, from 3 mm to 100 mm, from 3 mm to 90 mm, from 3 mm to 80 mm, from 3 mm to 70 mm, from 3 mm to 60 mm, from 3 mm to 50 mm, from 3 mm to 40 mm, from 5 mm to 110 mm, from 10 mm to 110 mm, from 20 mm to 110 mm, from 30 mm to 110 mm, from 40 mm to 110 mm, from 50 mm to 110 mm, from 60 mm to 110 mm, from 70 mm to 110 mm, from 80 mm to 110 mm, from 90 mm to 110 mm, or from 100 mm to 110 mm). In some embodiments, the loop of the electrode 130 and/or 140 has a circumferential length of at least about 3 mm, 10 mm, 25 mm, 50 mm cm, or 75 mm. In some embodiments, the loop of the electrode 130 and/or 140 has a circumferential length of at most about 110 mm, 100 mm, 90 mm, or 80 mm.

In some embodiments, the pair of electrodes 150 (e.g., 130 and 140), are separated by an electrode offset distance 133 (FIG. 5A). In general, the electrode offset determines the radius of the electric field which subsequently determines the effectiveness of phrenic nerve stimulation. In some embodiments, the offset 133 is created when an operator or control unit translates one electrode further distally than the other electrode of the pair of electrodes (e.g., 130 and 140). In some embodiments, the offset 133 is established during fabrication of the ETS system, wherein the pair of electrodes are secured to each other at a pre-determined offset prior to being inserted into the catheter lumen. In some embodiments, the offset is measured from the distal end of the proximal insulator 141 to the distal end of the distal insulator 131 (FIG. 5A) along the longitudinal axis of the catheter 120 (FIG. 5B). In some embodiments, the offset is measured from the distal end of the proximal electrode 140 loop to the distal end of the distal electrode 130 loop when the pair of electrodes 150 is in the unconstrained configuration (FIG. 5B). In some embodiments, the electrode offset 133 is measured in the constrained configuration. In some embodiments, the electrode offset 133 is measured in the unconstrained configuration. In some embodiments, the electrode offset 133 in the unconstrained configuration is approximately equal to the electrode offset 133 in the constrained configuration. In some embodiments, the electrode offset 133 in the unconstrained configuration is equal to the electrode offset 133 in the constrained configuration. In some embodiments, the electrode offset 133 in the unconstrained configuration is greater than the electrode offset 133 in the constrained configuration. In some embodiments, the electrode offset 133 in the unconstrained configuration is less than the electrode offset 133 in the constrained configuration. In some embodiments, the electrode offset is from about 0.25 cm to about 40 cm (e.g., about 0.25 cm, about 0.3 cm, about 0.4 cm, about 0.5 cm, about 0.6 cm, about 0.7 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, about 2.9 cm, about 3 cm, about 3.1 cm, about 3.2 cm, about 3.3 cm, about 3.4 cm, about 3.5 cm, about 3.6 cm, about 3.7 cm, about 3.8 cm, about 3.9 cm, about 4 cm, about 4.1 cm, about 4.2 cm, about 4.3 cm, about 4.4 cm, about 4.5 cm, about 4.6 cm, about 4.7 cm, about 4.8 cm, about 4.9 cm, about 5 cm, about 5.1 cm, about 5.2 cm, about 5.3 cm, about 5.4 cm, about 5.5 cm, about 5.6 cm, about 5.7 cm, about 5.8 cm, about 5.9 cm, about 6 cm, about 6.1 cm, about 6.2 cm, about 6.3 cm, about 6.4 cm, about 6.5 cm, about 6.6 cm, about 6.7 cm, about 6.8 cm, about 6.9 cm, about 7 cm, about 7.1 cm, about 7.2 cm, about 7.3 cm, about 7.4 cm, about 7.5 cm, about 7.6 cm, about 7.7 cm, about 7.8 cm, about 7.9 cm, about 8 cm, about 8.1 cm, about 8.2 cm, about 8.3 cm, about 8.4 cm, about 8.5 cm, about 8.6 cm, about 8.7 cm, about 8.8 cm, about 8.9 cm, about 9 cm, about 9.1 cm, about 9.2 cm, about 9.3 cm, about 9.4 cm, about 9.5 cm, about 9.6 cm, about 9.7 cm, about 9.8 cm, about 9.9 cm, about 10 cm, about 10.1 cm, about 10.2 cm, about 10.3 cm, about 10.4 cm, about 10.5 cm, about 10.6 cm about 10.7 cm, about 10.8 cm, about 10.9 cm, about 11 cm, about 11.1 cm, about 11.2 cm, about 11.3 cm, about 11.4 cm, about 11.5 cm, about 11.6 cm, about 11.7 cm, about 11.8 cm, about 11.9 cm, about 12 cm, about 12.1 cm, about 12.2 cm, about 12.3 cm, about 12.4 cm, about 12.5 cm, about 12.6 cm, about 12.7 cm, about 12.8 cm, about 12.9 cm, about 13 cm, about 13.1 cm, about 13.2 cm, about 13.3 cm, about 13.4 cm, about 13.5 cm, about 13.6 cm, about 13.7 cm, about 13.8 cm, about 13.9 cm, about 14 cm, about 14.1 cm, about 14.2 cm, about 14.3 cm, about 14.4 cm, about 14.5 cm, about 14.6 cm, about 14.7 cm, about 14.8 cm, about 14.9 cm, about 15 cm, about 15.1 cm, about 15.2 cm, about 15.3 cm, about 15.4 cm, about 15.5 cm, about 15.6 cm, about 15.7 cm, about 15.8 cm, about 15.9 cm, about 16 cm, about 16.1 cm, about 16.2 cm, about 16.3 cm, about 16.4 cm, about 16.5 cm, about 16.6 cm, about 16.7 cm, about 16.8 cm, about 16.9 cm, about 17 cm, about 17.1 cm, about 17.2 cm, about 17.3 cm, about 17.4 cm, about 17.5 cm, about 17.6 cm, about 17.7 cm, about 17.8 cm, about 17.9 cm, about 18 cm, about 18.1 cm, about 18.2 cm, about 18.3 cm, about 18.4 cm, about 18.5 cm, about 18.6 cm, about 18.7 cm, about 18.8 cm, about 18.9 cm, about 19 cm, about 19.1 cm, about 19.2 cm, about 19.3 cm, about 19.4 cm, about 19.5 cm, about 19.6 cm, about 19.7 cm, about 19.8 cm, about 19.9 cm, about 20 cm, about 20.1 cm, about 20.2 cm, about 20.3 cm, about 20.4 cm, about 20.5 cm, about 20.6 cm about 20.7 cm, about 20.8 cm, about 20.9 cm, about 21 cm, about 21.1 cm, about 21.2 cm, about 21.3 cm, about 21.4 cm, about 21.5 cm, about 21.6 cm, about 21.7 cm, about 21.8 cm, about 21.9 cm, about 22 cm, about 22.1 cm, about 22.2 cm, about 22.3 cm, about 22.4 cm, about 22.5 cm, about 22.6 cm, about 22.7 cm, about 22.8 cm, about 22.9 cm, about 23 cm, about 23.1 cm, about 23.2 cm, about 23.3 cm, about 23.4 cm, about 23.5 cm, about 23.6 cm, about 23.7 cm, about 23.8 cm, about 23.9 cm, about 24 cm, about 24.1 cm, about 24.2 cm, about 24.3 cm, about 24.4 cm, about 24.5 cm, about 24.6 cm, about 24.7 cm, about 24.8 cm, about 24.9 cm, about 25 cm, about 25.1 cm, about 25.2 cm, about 25.3 cm, about 25.4 cm, about 25.5 cm, about 25.6 cm, about 25.7 cm, about 25.8 cm, about 25.9 cm, about 26 cm, about 26.1 cm, about 26.2 cm, about 26.3 cm, about 26.4 cm, about 26.5 cm, about 26.6 cm, about 26.7 cm, about 26.8 cm, about 26.9 cm, about 27 cm, about 27.1 cm, about 27.2 cm, about 27.3 cm, about 27.4 cm, about 27.5 cm, about 27.6 cm, about 27.7 cm, about 27.8 cm, about 27.9 cm, about 28 cm, about 28.1 cm, about 28.2 cm, about 28.3 cm, about 28.4 cm, about 28.5 cm, about 28.6 cm, about 28.7 cm, about 28.8 cm, about 28.9 cm, about 29 cm, about 29.1 cm, about 29.2 cm, about 29.3 cm, about 29.4 cm, about 29.5 cm, about 29.6 cm, about 29.7 cm, about 29.8 cm, about 29.9 cm, about 30 cm, about 30.1 cm, about 30.2 cm, about 30.3 cm, about 30.4 cm, about 30.5 cm, about 30.6 cm about 30.7 cm, about 30.8 cm, about 30.9 cm, about 31 cm, about 31.1 cm, about 31.2 cm, about 31.3 cm, about 31.4 cm, about 31.5 cm, about 31.6 cm, about 31.7 cm, about 31.8 cm, about 31.9 cm, about 32 cm, about 32.1 cm, about 32.2 cm, about 32.3 cm, about 32.4 cm, about 32.5 cm, about 32.6 cm, about 32.7 cm, about 32.8 cm, about 32.9 cm, about 33 cm, about 33.1 cm, about 33.2 cm, about 33.3 cm, about 33.4 cm, about 33.5 cm, about 33.6 cm, about 33.7 cm, about 33.8 cm, about 33.9 cm, about 34 cm, about 34.1 cm, about 34.2 cm, about 34.3 cm, about 34.4 cm, about 34.5 cm, about 34.6 cm, about 34.7 cm, about 34.8 cm, about 34.9 cm, about 35 cm, about 35.1 cm, about 35.2 cm, about 35.3 cm, about 35.4 cm, about 35.5 cm, about 35.6 cm, about 35.7 cm, about 35.8 cm, about 35.9 cm, about 36 cm, about 36.1 cm, about 36.2 cm, about 36.3 cm, about 36.4 cm, about 36.5 cm, about 36.6 cm, about 36.7 cm, about 36.8 cm, about 36.9 cm, about 37 cm, about 37.1 cm, about 37.2 cm, about 37.3 cm, about 37.4 cm, about 37.5 cm, about 37.6 cm, about 37.7 cm, about 37.8 cm, about 37.9 cm, about 38 cm, about 38.1 cm, about 38.2 cm, about 38.3 cm, about 38.4 cm, about 38.5 cm, about 38.6 cm, about 38.7 cm, about 38.8 cm, about 38.9 cm, about 39 cm, about 39.1 cm, about 39.2 cm, about 39.3 cm, about 39.4 cm, about 39.5 cm, about 39.6 cm, about 39.7 cm, about 39.8 cm, about 39.9 cm, or about 40 cm). In some embodiments, the electrode offset 133 is at least about 0.5 cm, 1 cm, 2 cm, or 5 cm. In some embodiments, the electrode offset 133 is at most about 10 cm, 7 cm, or 5 cm.

In some embodiments, the pair of electrodes 150 contacts the trachea of a subject. In some embodiments, in the unconstrained configuration, the proximal electrode 140 and the distal electrode 130 contact the tracheal wall 015 of the subject (FIG. 5A) thereby enabling phrenic nerve pacing as described herein. In some embodiments, in the unconstrained configuration, the proximal electrode 140 and the distal electrode 130 are configured to contact at least about 30%, 40%, 50%, 60%, 70%, 80%, 90%, or 95% of the inner circumference of the tracheal wall of the subject. In some embodiments, in the unconstrained configuration the proximal electrode 140 and the distal electrode 130 contact the entire tracheal wall of the subject at a position along the proximal-distal axis of the trachea. In some embodiments, in the unconstrained configuration at least a portion of the proximal electrode 140) and the distal electrode 130 contact the tracheal tube of the subject.

In some embodiments, the proximal electrode 140) and the distal electrode 130 are made of the same conductive material. In some embodiments, the proximal electrode 140) and the distal electrode 130 are made of different conductive materials. In some embodiments, the conductive material is a material that is a conductor of electricity, which includes, for example, but is not limited to pure metal or alloys and is commonly used in the art for making wire electrodes. In some embodiments, the conductive material is selected from silicon, platinum, iridium, polyimide, gold, ceramics, nickel, titanium, copper, iron, chromium, or an alloy or combination thereof.

In some embodiments, the proximal electrode 140 and the distal electrode 130 are bipolar. In some embodiments, the bipolar electrodes enable pacing of the phrenic nerve (e.g., the left phrenic nerve 020, the right phrenic nerve 030, or both) that runs parallel to the trachea. In some embodiments, the bipolar electrodes enable pacing of the phrenic nerve that runs parallel to the trachea regardless of the angular orientation of the catheter 120 with respect to the trachea. In some embodiments, the proximal electrode 140 and the distal electrode 130 are monopolar. In some embodiments, in the unconstrained configuration at least a portion of the proximal electrode 140 and the distal electrode 130 extend beyond the ET tube 200.

In some embodiments, the one or more pairs of electrodes are configured to fit through the catheter 120 lumen. In general, each electrode has at least a portion (e.g., from about 1% to about 99% of the length of the electrode, e.g., from about 10% to about 99%, from about 20% to about 99%, from about 30% to about 99%, from about 40% to about 99%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 96% to about 99%, from about 97% to about 99%, or from about 98% to about 99% of the length of the electrode) covered by an insulator. In some embodiments, each electrode has at least a portion of 1% to 99% (e.g., from 10% to 99%, from 20% to 99%, from 30% to 99%, from 40% to 99%, from 50% to 99%, from 60% to 99%, from 70% to 99%, from 80% to 99%, from 85% to 99%, from 86% to 99%, from 87% to 99%, from 88% to 99%, from 89% to 99%, from 90% to 99%, from 91% to 99%, from 92% to 99%, from 93% to 99%, from 94% to 99%, from 95% to 99%, from 96% to 99%, from 97% to 99%, or from 98% to 99%) of the length of the electrode covered by the insulator. In some embodiments, the insulator is made of any material that resists the flow of electric current and/or having a resistivity of from about 10⁷ Ω·m to about 10¹⁵ Ω·m (e.g., from about 10⁸ Ω·m to about 10¹⁵ Ω·m, from about 10⁹ Ω·m to 10¹⁴ Ω·m, from about 10¹⁰ Ω·m to about 10¹³ Ω·m, or from about 10¹⁰ Ω·m to about 10¹² Ω·m). In some embodiments, the insulator is made of any material that resists the flow of electric current and/or having a resistivity of at least about 10⁷ Ω·m, 10⁸ Ω·m, 10⁹ Ω·m, 10¹⁰ Ω·m, 10¹¹ Ω·m, 10¹² Ω·m, 10¹³ Ω·m, 10¹⁴ Ω·m, or 10¹⁵ Ω·m. In some embodiments, the insulator is made of any material that resists the flow of electric current and/or having a resistivity of at least about 10¹⁰ Ω·m. Examples of suitable insulator materials may include, but are not limited to, a polymer insulator (such as silicone, polyurethane, polytetrafluoroethylene (e.g., TEFLON™), or other fluoropolymers), a ceramic insulator, or a glass insulator. An insulative coating enables the control, direction, and focus of the stimulation signal delivered by the electrodes to the phrenic nerves. An insulative coating also allows dividing the electrode into multiple electrode stimulation regions for optimizing the stimulation location and/or for operating in a multi-electrode configuration, such as a bipolar or tripolar electrode. In some embodiments, the electrode insulator 141 and/or 131 (FIG. 4C) has an outer diameter of from about 0.5 mm to about 10 mm (e.g., about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9) mm, about 9.1 mm, about 9.2 mm, about 9.3 mm, about 9.4 mm, about 9.5 mm, about 9.6 mm, about 9.7 mm, about 9.8 mm, about 9.9 mm, or about 10 mm).

### Catheter

In some embodiments, the catheter 120 is configured to fit through the guide sheath 110 lumen. In some embodiments, the catheter 120 protrudes from the guide sheath 110. In some embodiments, the catheter 120 is longitudinally translatable with respect to the ET tube 200. In some embodiments, the catheter 120 has an outer diameter of from about 1.5 mm to about 20 mm (e.g., about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm). In some embodiments, the catheter 120 has a lumen, the lumen having a diameter of from about 1.3 mm to about 19.8 mm (e.g., about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 19.5 mm, or about 19.8 mm).

In some embodiments, the catheter 120 has a length from a proximal end to a distal end of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the catheter 120 has a length from a proximal end to a distal end of from 1 cm to 100 cm (e.g., from 10 cm to 100 cm, from 10 cm to 90 cm, from 10 cm to 80 cm, from 10 cm to 70 cm, from 10 cm to 60 cm, from 10 cm to 50 cm, from 20 cm to 100 cm, from 30 cm to 100 cm, from 40 cm to 100 cm, from 50 cm to 100 cm, from 60 cm to 100 cm, from 70 cm to 100 cm, from 80 cm to 100 cm, or from 90 cm to 100 cm). In some embodiments, the catheter 120 is flexible, pliable, or both. In some embodiments, a length of the catheter 120 maintains its length during use.

In some embodiments, the distal portion of the catheter 120 is biased to have a shape that is non-straight. In some embodiments, the catheter 120 is formed of a shape memory material (e.g., nitinol) where the non-straight shape is achieved by actuating the shape memory material through electricity, heat, a chemical reaction, or any combination thereof.

In some embodiments, each electrode pair 150 is attached to the catheter 120 lumen and is translatable through the guide sheath 110 together with the catheter 120. In some embodiments, each electrode pair 150 is not attached to the catheter 120 lumen and is translatable through the catheter 120 lumen along the longitudinal axis of the catheter 120.

### Guide Sheath

The guide sheath 110 is configured to fit through the ET tube 200 lumen. In some embodiments, the guide sheath 110 is translatable along a longitudinal axis with respect to the ET tube 200. In some embodiments, the guide sheath 110 has an outer diameter of from about 1.5 mm to about 20 mm (e.g., from about 1.5 mm to about 15 mm, from about 1.5 mm to about 10 mm, from about 1.5 mm to about 5 mm, or from about 1.5 mm to about 2.5 mm). In some embodiments, the guide sheath 110 has a lumen, the lumen having a diameter of from about 1.3 mm to about 19.8 mm (e.g., from about 1.3 mm to about 15 mm, from about 1.3 mm to about 10 mm, from about 1.3 mm to about 5 mm, from about 1.3 mm to about 2.5 mm, or from 1.3 mm to about 2.2 mm). In some embodiments, the guide sheath 110 lumen surrounds at least a portion of the catheter 120.

In some embodiments, the pair of electrodes 150 translate together within the guide sheath 110. In some embodiments, the pair of electrodes 150 translate independently within the guide sheath 110. In some embodiments, the guide sheath 110 retracts from the pair of electrodes 150.

In some embodiments, the guide sheath 110 has a length from a proximal end to a distal end of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the guide sheath 110 has a length from a proximal end to a distal end of from 1 cm to 100 cm (e.g., from 10 cm to 100 cm, from 10 cm to 90 cm, from 10 cm to 80 cm, from 10 cm to 70 cm, from 10 cm to 60 cm, from 10 cm to 50 cm, from 20 cm to 100 cm, from 30 cm to 100 cm, from 40 cm to 100 cm, from 50 cm to 100 cm, from 60 cm to 100 cm, from 70 cm to 100 cm, from 80 cm to 100 cm, or from 90 cm to 100 cm). In some embodiments, the length of the guide sheath 110 is longer than the length of the ET tube 200.

### ET Tube, ET Tube Connector, and Sensor

The ETS system, in general, includes an ET tube 200. The ET tube 200 is configured to be inserted into a subject's trachea 010 (FIG. 2). In some embodiments, inserting the ET tube into a subject's trachea 010 protects the subject's airway and provides ventilation during the pacing of the phrenic nerves 020 and/or 030 of the subject. In some embodiments, the ET tube is an oral ET tube 200. In some embodiments, the ET tube is a nasal ET tube. In some embodiments, the ET tube is a tracheostomy tube. In some embodiments, the ET tube is configured to function with all embodiments and/or variations of the guide sheath 110, catheter 120, ET tube connector 300, or the one or more pairs of electrodes herein described.

In some embodiments, the ET tube 200 has an outer diameter of from about 3 mm to about 20 mm (e.g., from about 3 mm to about 18 mm, from about 4 mm to about 16 mm, from about 5 mm to about 14 mm, from about 6 mm to about 13 mm, from about 7 mm to about 12 mm, or from about 8 mm to about 11 mm). In some embodiments, the ET tube 200 has an outer diameter of at most 20 mm (e.g., at most 15, at most 10 mm, at most 5 mm). In some embodiments, the ET tube 200 has a lumen, the lumen having a diameter of from about 2.5 mm to about 19.8 mm (e.g., from about 2.5 mm to about 15 mm, from about 2.8 mm to about 12 mm, from about 2.8 mm to about 10 mm, or from about 5 mm to about 10 mm).

The ET tube 200, in some embodiments, has a length from a proximal end to a distal end of from 12 cm to 40 cm (e.g., about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm).

In some embodiments, the ETS platform includes an ET tube connector 300. The ET tube connector 300, in some embodiments, bridges the proximal end of the ET tube and the distal end of the ventilator tubing circuit 410 (FIG. 2). In some embodiments, the ET tube connector 300 includes a port 340 (FIG. 3). In some embodiments, the port 340 provides an entry point for the guide sheath 110, the catheter 120, and the electrodes 150 to enter the ET tube 200 via the proximal end of the ET tube 200 (FIG. 3).

In some embodiments, the ET tube connector includes a sensor 310 (FIG. 2). In some embodiments, the sensor is capable of measuring one or more parameters including air flow, pressure, or temperature. In some embodiments, the control unit 500 is in electrical communication with the sensor 310.

### Second Endotracheal Stimulation System

In another aspect, the ETS platform 1000 provided herein includes a second ETS system 600 as shown in FIGS. 6A-6C. The second ETS system 600 may, in some embodiments, include an ET tube 200, an ET tube connector 300, a guide sheath 610, a catheter 620 and one or more pairs of electrodes 633. FIG. 6A shows an exemplary second endotracheal stimulation system 600 in a constrained configuration. The catheter 620 and the guide sheath 610 are shown extending through the distal opening of the endotracheal tube 202.

In some embodiments, the second ETS system 600 is configured to engage with one or more of the systems of the ETS platform 1000 including the one or more data cables 320, the control unit 500, the ventilator 400, and the ventilator tubing 410. In some embodiments, a locking mechanism 330, similar to that shown in FIG. 3 is configured to be used with the second ETS system 600 to secure the data cable 320 to the endotracheal tube connector via the endotracheal tube connector port 340. In some embodiments, one or more components of the second ETS system 600 are configured to be located within an ET tube lumen 205 (FIG. 6A), wherein the ET tube 200 is configured to be inserted into the trachea of a subject.

In some embodiments, the second ETS system 600 has a constrained configuration as shown in FIG. 6A, wherein the catheter 620 is in an initial configuration useful for translating the catheter through the lumen of an ET tube 205 from a proximal end of the ET tube 200 to the distal opening of the ET tube 202 (FIG. 6A). In some embodiments, in the constrained configuration, the distal portion of the catheter 625 is within the guide sheath 610 (FIG. 6A). In some embodiments, the second ETS system 600 has an unconstrained or deployed configuration as seen in FIG. 6B and FIG. 6C. In some embodiments, in the unconstrained configuration, the distal portion of the catheter 625 extends out of the guide sheath 610 (FIG. 6B).

In some embodiments, the catheter 620 is configured to self-arrange into a predetermined shape when fully deployed through the ET tube 200.

### Electrodes

In some embodiments, at least a distal region of the catheter 620 comprises one or more pairs of electrodes 630. In some embodiments, the pair of electrodes 630 are in electrical communication with a proximal portion of the system 600. In some embodiments, the pair of electrodes 630 includes a distal electrode 631 and a proximal electrode 632. In some embodiments, the one or more pairs of electrodes 630 are in electrical communication with a proximal portion of the system 600 for connection to the control unit 500. In some embodiments, the electrical communication is wired, wireless, or both. In some embodiments, the wired electrical communication employs a locking mechanism 330 to ensure that electrical communication is maintained throughout the operation of the ETS system 600.

FIG. 6B shows a side-view of an exemplary second ETS system 600 in the unconstrained configuration. The catheter 620 is shown having multiple pairs of electrodes 630. Each pair of electrodes 630 shown includes a distal electrode 631 and a proximal electrode 632. The guide sheath 610 is shown extending through the opening at the distal end of the endotracheal tube 200. In some embodiments, the one or more pairs of electrodes 630 are integral to the catheter 620. In some embodiments, the one or more pairs of electrodes are fastened to the catheter. In some embodiments, the one or more pairs of electrodes are disposed on an outer surface of the catheter 620 (FIG. 6B-6C). In some embodiments, the catheter 620 is shaped as a flexible cylindrical tube and the one or more pairs of electrodes 630 fully encircle separate sections of the catheter at varying positions of the catheter as shown in FIG. 6B.

In FIG. 6C a top view of an exemplary distal region 625 of a catheter 620 and four pairs of electrodes 630 disposed along the length of the catheter 620 are shown in the unconstrained configuration. The catheter 620 is shaped as a loop having a catheter outer diameter 660. The distance from an outer edge of a first pair of electrodes 630 to the outer edge of a second pair of electrodes is shown as the electrode outer diameter 670. A distance along the length of the catheter 620 and between a distal electrode 631 and a proximal electrode 632 is shown as the electrode offset 633. A distance along the length of the catheter 620 that separates a first pair of electrodes from a second pair of electrodes is shown as the electrode pair offset 635. In some embodiments, a distance between a distal electrode 631 and a proximal electrode 632 is less than a distance between the proximal electrode 632 and a distal electrode of a subsequent pair of electrodes. In some embodiments, a distance between a distal electrode 631 and a proximal electrode 632 is greater than a distance between the proximal electrode 632 and a distal electrode of a subsequent pair of electrodes.

In some embodiments, as shown in FIGS. 6B and 6C, the pairs of electrodes 630 extend along the length of the distal portion of the catheter 625. In some embodiments, the pairs of electrodes 630 extend along at most a portion of the distal portion of the catheter 625. In some embodiments, one or more electrodes are located at a tip of the catheter 620. In some embodiments, the pair of electrodes 630 are bipolar. In some embodiments, the bipolar electrodes enable pacing of the phrenic nerve that runs parallel to the trachea 010. In some embodiments, the bipolar electrodes enable pacing of the phrenic nerve that runs parallel to the trachea regardless of the angular orientation of the catheter 620 with respect to the trachea 010. In some embodiments, the pair of electrodes 630 are monopolar.

In some embodiments, each of the one or more pairs of electrodes 630 of the second ETS system 600 includes a distal electrode 631 (FIGS. 6B-6C) and a proximal electrode 632 (FIGS. 6B-6C), wherein the distal electrode 631 is disposed more distal to the proximal electrode 632 along the length of the catheter 620 from the catheter proximal end to the distal region of the catheter 625.

In some embodiments, each electrode in the one or more pairs of electrodes 630 has a length 640 of from about 1 mm to about 10 mm (e.g., from about 1 mm to about 9 mm, from about 1 mm to about 8 mm, from about 1 mm to about 7 mm, from about 1 mm to about 6 mm, or from about 1 mm to about 5 mm). In some embodiments, each electrode, in the one or more pairs of electrodes 630, has a length of at least about 1 mm (e.g., at least about 2 mm, at least about 3 mm, at least about 4 mm, or at least about 5 mm) along the length of the catheter 630 (FIG. 6C). In some embodiments, each electrode, in the one or more pairs of electrodes 630, has a length of at most about 10 mm (e.g., at most about 9 mm, at most about 8 mm, at most about 7 mm, at most about 6 mm, or at most about 5 mm) along the length of the catheter 630 (FIG. 6C). In some embodiments, the one or more pairs of electrodes have a different length 640). In some embodiments, each electrode has a unique length 640.

In some embodiments, each electrode in the one or more pairs of electrodes 630 has a width 650 of from about 1 mm to about 10 mm (e.g., from about 1 mm to about 9 mm, from about 1 mm to about 8 mm, from about 1 mm to about 7 mm, from about 1 mm to about 6 mm, or from about 1 mm to about 5 mm). In some embodiments, each electrode, in the one or more pairs of electrodes 630, has a width of at least about 1 mm (e.g., at least about 2 mm, at least about 3 mm, at least about 4 mm, or at least about 5 mm) along the width of the catheter 630) (FIG. 6C). In some embodiments, each electrode, in the one or more pairs of electrodes 630, has a width of at most about 10 mm (e.g., at most about 9 mm, at most about 8 mm, at most about 7 mm, at most about 6 mm, or at most about 5 mm) along the width of the catheter 630 (FIG. 6C). In some embodiments, the one or more pairs of electrodes have a different width 650. In some embodiments, each electrode has a unique width 650. In some embodiments, one or more electrodes of the ETS 600 have substantially the same length 640 and width 650. In some embodiments, one or more electrodes of the ETS 600 have a larger length 640) than width 650. In some embodiments, one or more electrodes of the ETS 600 have smaller length 640 than width 650.

In some embodiments, the distance between a distal electrode 631 and a proximal electrode 632, along the length of the catheter 620, referred herein as the electrode offset 633 (FIG. 6C), is from about 2 mm to about 10 mm (e.g., from about 2 mm to about 8 mm, from about 2 mm to about 6 mm, from about 2 mm to about 4 mm, from about 4 mm to about 8 mm, or from about 6 mm to about 8 mm). In some embodiments, the offset 633 is established during fabrication of the ETS system, wherein the one or more pairs of electrodes 630 are secured to the catheter 620. In some embodiments, the electrode offset is measured from the distal end of the distal electrode 631 to the proximal end of the proximal electrode 632 along the longitudinal axis of the catheter 620. In some embodiments, the electrode offset is measured from the proximal end of the distal electrode 631 to the distal end of the proximal electrode 632 along the longitudinal axis of the catheter 620. In some embodiments, the electrode offset is measured from the distal end of the distal electrode 631 to the distal end of the proximal electrode 632 along the longitudinal axis of the catheter 620. In some embodiments, the electrode offset is measured from the proximal end of the distal electrode 631 to the proximal end of the proximal electrode 632 along the longitudinal axis of the catheter 620. In some embodiments, the electrode offset is measured from the center of the distal electrode 631 to the center of the proximal electrode 632 along the longitudinal axis of the catheter 620.

In some embodiments, the distance from a first electrode pair to a second electrode pair, along the length of the catheter 620, referred herein as the electrode pair offset 635 (FIG. 6C) is from about 0.25 cm to about 16 cm (e.g., from about 0.5 cm to about 15 cm, from about 0.75 cm to about 10 cm, or from about 1 cm to about 5 cm). In some embodiments, the electrode pair offset is measured from the distal end of the distal electrode 631 of a distal electrode pair to the proximal end of the proximal electrode 632 of a proximal electrode pair along the length of the catheter 620. In some embodiments, the electrode pair offset is measured from the proximal end of the proximal electrode 631 of a distal electrode pair to the distal end of the distal electrode 632 of a proximal electrode pair along the length of the catheter 620). In some embodiments, the electrode pair offset is measured from the distal end of the distal electrode 631 of a distal electrode pair to the distal end of the distal electrode 632 of a proximal electrode pair along the length of the catheter 620. In some embodiments, the electrode pair offset is measured from the proximal end of the proximal electrode 631 of a distal electrode pair to the proximal end of the proximal electrode 632 of a proximal electrode pair along the length of the catheter 620. In some embodiments, the electrode pair offset is measured from the center of the distal electrode pair to the center of the proximal electrode pair along the longitudinal axis of the catheter 620. In some embodiments, the plurality of pairs of electrodes 630 are evenly spaced apart along the length of the distal region of the catheter 625. In some embodiments, the plurality of pairs of electrodes 630 are not evenly distributed along the length of the distal region of the catheter 625.

In some embodiments, the proximal electrode 632 and/or the distal electrode 631 have a width larger than the width of the catheter 620 (FIG. 6C). In some embodiments, the larger width of an electrode compared to the width of the catheter 620 provides the electrode with an outer edge 651 and an inner edge 652 (FIG. 6C). In some embodiments, the diameter of the distal region of the catheter 625 is measured from the outer edge 651 of a first electrode to an outer edge of a second electrode positioned opposite to the first electrode relative to the loop of the distal region of the catheter 625 (FIG. 6C). In some embodiments, the electrode outer diameter 670) is from about 10 mm to about 40 mm (e.g., from about 10 mm to about 30 mm, from about 10 mm to about 20 mm, e.g., at most about 30 mm, at most about 25 mm, at most about 20 mm, or at most about 15 mm).

In some embodiments, the one or more pairs of electrodes 630 contact the trachea of a subject. In some embodiments, in the unconstrained configuration, the one or more pairs of electrodes 630 contact the tracheal wall 015 of the subject (FIG. 5A) thereby enabling phrenic nerve pacing as described herein. In some embodiments, in the unconstrained configuration, the one or more pairs of electrodes 630) are configured to contact at least about 10%, 20%, 30%, 40%, 50%, 60%, 70%, 80%, 90%, or 95% of the inner circumference of the tracheal wall of the subject. In some embodiments, in the unconstrained configuration, the proximal electrode 632 and the distal electrode 631 contact the entire tracheal wall of the subject at a position along the proximal-distal axis of the trachea. In some embodiments, in the unconstrained configuration, at least a portion of the one or more pairs of electrodes 630 contact the tracheal wall 015 of the subject.

In some embodiments, the one or more pairs of electrodes 630 are made of the same conductive material. In some embodiments, the one or more pairs of electrodes 630 are made of different conductive materials. In some embodiments, the conductive material is a material that is a conductor of electricity, which includes, for example, but is not limited to pure metal or alloys and is commonly used in the art for making wire electrodes. In some embodiments, the conductive material is selected from silicon, platinum, iridium, polyimide, gold, ceramics, nickel, titanium, copper, iron, chromium, or an alloy or combination thereof.

In some embodiments, the one or more pairs of electrodes 630 are bipolar. In some embodiments, the bipolar electrodes enable pacing of the phrenic nerve (e.g., the left phrenic nerve 020, the right phrenic nerve 030, or both) that runs parallel to the trachea 015 (FIG. 1). In some embodiments, the bipolar electrodes enable pacing of the phrenic nerve that runs parallel to the trachea 015 regardless of the angular orientation of the catheter 620 with respect to the trachea 015. In some embodiments, the one or more pairs of electrodes 630 are monopolar. In some embodiments, in the unconstrained configuration, at least a portion of the proximal electrode 632 and the distal electrode 631 extend beyond the ET tube 200.

### Catheter

In some embodiments, the catheter 620 is configured to fit through the guide sheath 610 lumen. In some embodiments, the catheter 620 protrudes from the guide sheath 610. In some embodiments, the catheter 620 is longitudinally translatable with respect to the ET tube 200. In some embodiments, the catheter 620 has an outer diameter of from about 1 mm to about 35 mm (e.g., about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9) mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, or about 35 mm). In some embodiments, the catheter 620 has a lumen, the lumen having a diameter of from about 1.3 mm to about 34.8 mm (e.g., about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9) mm, about 10 mm, about 11 mm, about 12 mm, about 13 mm, about 14 mm, about 15 mm, about 16 mm, about 17 mm, about 18 mm, about 19 mm, about 20, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 34.5 mm, or about 34.8 mm).

In some embodiments, the catheter 620 has a length from a proximal end to a distal end of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the catheter 620 has a length from a proximal end to a distal end of from 1 cm to 100 cm (e.g., from 10 cm to 100 cm, from 10 cm to 90 cm, from 10 cm to 80 cm, from 10 cm to 70 cm, from 10 cm to 60 cm, from 10 cm to 50 cm, from 20 cm to 100 cm, from 30 cm to 100 cm, from 40 cm to 100 cm, from 50 cm to 100 cm, from 60 cm to 100 cm, from 70 cm to 100 cm, from 80 cm to 100 cm, or from 90 cm to 100 cm). In some embodiments, the catheter 620 is flexible, pliable, or both. In some embodiments, a length of the catheter 620 maintains its length during use.

The catheter 620 is configured to carry one or more wires that electrically connect the one or more pairs of electrodes disposed at the distal portion of the catheter 625 and the control unit 500.

In some embodiments, the catheter 620 is shaped as a loop (FIG. 6B-6C). In some embodiments the catheter loop has a diameter 660 of from 1.6 mm to 30 mm (e.g., about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, or about 30) mm). In some embodiments, the catheter loop has a diameter 660 of at least about 1 cm, 5 cm, 10 cm, 15 cm, or 20 cm. In some embodiments, the catheter loop has a diameter 660 of at most about 35 cm, 30 cm, or 25 cm. In some embodiments, the catheter loop diameter 660 is measured from an outer edge of the loop to an outer edge on the opposite side of the catheter loop (i.e., an outer diameter) (FIG. 6C). In some embodiments, the catheter loop diameter 660 is measured from an inner edge of the loop to an inner edge on the opposite side of the catheter loop (i.e., an inner diameter) (FIG. 6C).

In some embodiments, the distal portion of the catheter 625 is biased to have a shape that is non-straight. In some embodiments, the catheter 620 is formed of a shape memory material (e.g., nitinol) where the non-straight shape is achieved by actuating the shape memory material through electricity, heat, a chemical reaction, or any combination thereof. In some embodiments, the catheter 620 has a non-straight shape in the unconstrained configuration with a minimum, average, or maximum arc angle in one or more directions of from about 90 degrees to about 360 degrees (e.g., from about 100 degrees to about 360) degrees, from about 110 degrees to about 360 degrees, from about 120 degrees to about 360 degrees, from about 130 degrees to about 360 degrees, from about 140 degrees to about 360) degrees, from about 150 degrees to about 360 degrees, from about 160 degrees to about 360 degrees, from about 170 degrees to about 360 degrees, from about 180 degrees to about 360 degrees, from about 190 degrees to about 360 degrees, from about 200 degrees to about 360 degrees, from about 210) degrees to about 360 degrees, from about 220 degrees to about 360 degrees, from about 230 degrees to about 360 degrees, from about 240 degrees to about 360 degrees, from about 250) degrees to about 360 degrees, from about 260 degrees to about 360) degrees, from about 270) degrees to about 360 degrees, from about 280 degrees to about 360 degrees, from about 290 degrees to about 360 degrees, from about 300 degrees to about 360 degrees, from about 310 degrees to about 360 degrees, from about 320 degrees to about 360 degrees, from about 330 degrees to about 360 degrees, from about 340 degrees to about 360 degrees, from about 350 degrees to about 360 degrees). In some embodiments, the catheter has an arc angle in one or more directions of at least about 100 degrees, at least about 120 degrees, at least about 150 degrees, at least about 180 degrees, at least about 210 degrees, at least about 240, at least about 260 degrees, or at least about 300 degrees). In some embodiments, the catheter 620 has a non-straight shape in the unconstrained configuration with a minimum, average, or maximum arc angle in one or more directions of at least about 90 degrees, 120 degrees, 150 degrees, 180 degrees, 210 degrees, 240 degrees, 270 degrees, 300 degrees, or 330 degrees, including increments therein.

In some embodiments, the catheter loop has a circumferential length (e.g., from one end of the loop to the other end of the loop) of from about 3 mm to about 110 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40) mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, or about 110 mm). In some embodiments, the catheter loop has a circumferential length of from 3 mm to 110 mm (e.g., from 5 mm to 100 mm, from 10 mm to 90 mm, from 20 mm to 80 mm, from 30 mm to 70 mm, from 40 mm to 60 mm, from 3 mm to 100 mm, from 3 mm to 90 mm, from 3 mm to 80 mm, from 3 mm to 70 mm, from 3 mm to 60 mm, from 3 mm to 50 mm, from 3 mm to 40 mm, from 5 mm to 110 mm, from 10 mm to 110 mm, from 20 mm to 110 mm, from 30 mm to 110 mm, from 40 mm to 110 mm, from 50 mm to 110 mm, from 60 mm to 110 mm, from 70 mm to 110 mm, from 80 mm to 110 mm, from 90 mm to 110 mm, or from 100 mm to 110 mm). In some embodiments, the catheter loop has a circumferential length of at least about 3 mm, 10 mm, 25 mm, 50 mm cm, or 75 mm. In some embodiments, the catheter loop has a circumferential length of at most about 110 mm, 100 mm, 90 mm, or 80 mm.

### Guide Sheath

The guide sheath 610 is configured to fit through the ET tube lumen 205 as seen in FIG. 6A. In some embodiments, the guide sheath 610 is translatable along a longitudinal axis with respect to the ET tube 200. In some embodiments, the guide sheath 610 has an outer diameter of from about 1.5 mm to about 20 mm (e.g., from about 1.5 mm to about 15 mm, from about 1.5 mm to about 10 mm, from about 1.5 mm to about 5 mm, or from about 1.5 mm to about 2.5 mm). In some embodiments, the guide sheath 110 has a lumen, the lumen having a diameter of from about 1.3 mm to about 19.8 mm (e.g., from about 1.3 mm to about 15 mm, from about 1.3 mm to about 10 mm, from about 1.3 mm to about 5 mm, from about 1.3 mm to about 2.5 mm, or from 1.3 mm to about 2.2 mm). In some embodiments, the guide sheath 610 lumen surrounds at least a portion of the catheter 620.

In some embodiments, the guide sheath 610 has a length from a proximal end to a distal end of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the guide sheath 610 has a length from a proximal end to a distal end of from 1 cm to 100 cm (e.g., from 10 cm to 100 cm, from 10 cm to 90 cm, from 10 cm to 80 cm, from 10 cm to 70 cm, from 10 cm to 60 cm, from 10 cm to 50 cm, from 20 cm to 100 cm, from 30 cm to 100 cm, from 40 cm to 100 cm, from 50 cm to 100 cm, from 60 cm to 100 cm, from 70 cm to 100 cm, from 80 cm to 100 cm, or from 90 cm to 100 cm). In some embodiments, the length of the guide sheath 610 is longer than the length of the ET tube 200.

### ET Tube, ET Tube Connector and Sensor

The ETS system, in general, includes an ET tube 200. The ET tube 200 is configured to be inserted into a subject's trachea 010 (FIG. 2). In some embodiments, inserting the ET tube into a subject's trachea 010 protects the subject's airway and provides ventilation during the pacing of the phrenic nerves 020 and/or 030 of the subject. In some embodiments, the ET tube is an oral ET tube 200. In some embodiments, the ET tube is a nasal ET tube. In some embodiments, the ET tube is a tracheostomy tube. In some embodiments, the ET tube is configured to function with all embodiments and/or variations of the guide sheath 610, catheter 620, ET tube connector 300, or the one or more pairs of electrodes 630 herein described.

In some embodiments, the ET tube 200 has an outer diameter of from about 3 mm to about 20 mm (e.g., from about 3 mm to about 18 mm, from about 4 mm to about 16 mm, from about 5 mm to about 14 mm, from about 6 mm to about 13 mm, from about 7 mm to about 12 mm, or from about 8 mm to about 11 mm). In some embodiments, the ET tube 200 has an outer diameter of at most 20 mm (e.g., at most 15, at most 10 mm, at most 5 mm). In some embodiments, the ET tube 200 has a lumen, the lumen having a diameter of from about 2.8 mm to about 19.8 mm (e.g., from about 2.8 mm to about 15 mm, from about 2.8 mm to about 10 mm, or from about 2.8 mm to about 5 mm).

The ET tube 200, in some embodiments, has a length from a proximal end to a distal end of from 12 cm to 40 cm (e.g., about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm).

In some embodiments, the ETS platform includes an ET tube connector 300. The ET tube connector 300, in some embodiments, bridges the proximal end of the ET tube and the distal end of the ventilator tubing circuit 410 (FIG. 2). In some embodiments, the ET tube connector 300 includes a port 340 (FIG. 3). In some embodiments, the port 340 provides an entry point for the guide sheath 610, the catheter 620, and the one or more pairs of electrodes 630 to enter the ET tube 200 via the proximal end of the ET tube 200 (FIG. 3).

In some embodiments, the ET tube connector includes a sensor 310 (FIG. 2). In some embodiments, the sensor is capable of measuring one or more parameters including air flow, pressure, or temperature. In some embodiments, the control unit 500 is in electrical communication with the sensor 310.

### Third Endotracheal Stimulation System

In another aspect, the ETS platform 1000 provided herein includes a third ETS system 700 as shown in FIGS. 7-8D. The third ETS system 700 may, in some embodiments, include an ET tube 200, an ET tube connector 300, a catheter 720, and one or more pairs of electrodes 733. FIG. 7 shows an exemplary third ETS system 700 where the endotracheal tube 200 includes an endotracheal tube cuff 210 at the distal end of the endotracheal tube 202, a Murphy's eye 220, and an opening 204 at the distal end of the endotracheal tube. A catheter 720, shaped as a spiral catheter, is shown having a plurality of electrode pairs 730. In FIG. 7, the catheter 720 is shown configured to wrap around the endotracheal tube 200 from the proximal end of the endotracheal tube 201 to the distal end of the endotracheal tube 202. Each pair of electrodes 730 contains a distal electrode 731 and a proximal electrode 732. The distal electrode 731 and the proximal electrode 732 are separated by an electrode offset 733 distance along the length of the catheter 720. In some embodiments, the third ETS system includes a pilot balloon line 215 as shown in FIG. 7.

In some embodiments, the third ETS system 700 is configured to engage with one or more of the systems of the ETS platform 1000 including the one or more data cables 320, the control unit 500, the ventilator 400, and the ventilator tubing 410. In some embodiments, a locking mechanism 330, similar to that shown in FIG. 3 is configured to be used with the third ETS system 700 to secure the data cable 320 to the endotracheal tube connector via the endotracheal tube connector port 340. In some embodiments, one or more components of the third ETS system 700 are configured to be disposed around an outer surface of the ET tube 200 (FIGS. 7-8D), wherein the ET tube 200 is configured to be inserted into the trachea of a subject.

### Electrodes

In some embodiments, at least a portion of the catheter 720 includes one or more pairs of electrodes 730. In some embodiments, the pair of electrodes 730 are in electrical communication with a proximal portion of the ETS system 700. In some embodiments, the pair of electrodes 730 includes a distal electrode 731 and a proximal electrode 732, wherein the distal electrode 731 is disposed more distal to the proximal electrode 732 along the length of the catheter 720 from the proximal end of the catheter to the distal end of the catheter. In some embodiments, the one or more pairs of electrodes 730 are in electrical communication with a proximal portion of the system 700 for connection to the control unit 500. In some embodiments, the electrical communication is wired, wireless, or both. In some embodiments, the wired electrical communication employs a locking mechanism 330 to ensure that electrical communication is maintained throughout the operation of the ETS system 700. In some embodiments, the catheter includes a group of three electrodes, as shown in FIG. 8B, wherein a distal electrode 731, a proximal electrode 732, and a middle electrode 734 are disposed on the catheter 720.

In some embodiments, the one or more pairs of electrodes 730 are located on an outer surface of the catheter 720. In some embodiments, the pair of electrodes 730 are bipolar. In some embodiments, the bipolar electrodes enable pacing of the phrenic nerve that runs parallel to the trachea regardless of the angular orientation of the inner catheter with respect to the trachea 010. In some embodiments, the pair of electrodes 730 are monopolar. In some embodiments, the one or more pairs of electrodes 730 are evenly distributed along the length of the electrode 720. In some embodiments, the one or more pairs of electrodes 730 are not evenly distributed along the length of the catheter 720. In some embodiments, the pair of electrodes 730) are arranged longitudinally along the collar-shaped catheter along an axis parallel to that of the ET tube 200 (FIGS. 8A-8D).

In some embodiments, the one or more pairs of electrodes 730 have a circular shape as shown in FIG. 7. In some embodiments, the one or more pairs of electrodes are rectangular shaped as seen in FIGS. 8A-8D. In some embodiments, the one or more pairs of electrodes have a circular, a rectangular, a triangular, an oval, a D-shape, a star shape, a polygonal shape, or any combination or variation thereof.

In some embodiments, the one or more pairs of electrodes 730, when shaped in a circular configuration (FIG. 7), have a diameter of from about 1 mm to 10 mm (e.g., from about 2 mm to 10 mm, from 3 mm to about 10 mm, from about 4 mm to about 10 mm, from about 5 mm to about 10 mm, or from about 6 mm to about 10 mm). In some embodiments, the one or more pairs of electrodes 730 have a diameter of at least about 1 mm, about 2 mm, about 3 mm, about 4 mm, or about 5 mm. In some embodiments, the one or more pairs of electrodes have a diameter of at most about 10 mm, about 9 mm, about 8 mm, about 7 mm, or about 6 mm.

In some embodiments, the one or more pairs of electrodes 730, when shaped in a rectangular configuration (FIGS. 8A-8B), have a length and a width wherein the length is the dimension of the electrode that extends around the circumference of the ET tube and the width is the dimension of the electrode from the proximal end of the ET tube to the distal end of the ET tube. In some embodiments, the length of an electrode of the one or more pairs of electrodes is from about 3 mm to about 110 mm (e.g., about 3 mm, about 4 mm, about 5 mm, about 6 mm, about 7 mm, about 8 mm, about 9) mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40) mm, about 41 mm, about 42 mm, about 43 mm, about 44 mm, about 45 mm, about 46 mm, about 47 mm, about 48 mm, about 49 mm, about 50) mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70) mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, or about 110 mm).

In some embodiments, the width of an electrode of the one or more pairs of electrodes 730 is from about 1 mm to about 20 mm (e.g., from about 2 mm to about 15 mm, or from about 5 mm to about 10 mm). In some embodiments, the width of an electrode of the one or more pairs of electrodes 730 is at least about 1 mm, about 2 mm, about 5 mm, about 10 mm, or about 15 mm. In some embodiments, the width of an electrode of the one or more pairs of electrodes 730 is at most about 20 mm, about 15 mm, about 10 mm, or about 5 mm.

In some embodiments, a distance between the distal electrode 731 and a proximal electrode 732 is less than a distance between a proximal electrode 732 and a subsequent distal electrode 731. In some embodiments, a distance between the distal electrode 731 and a proximal electrode 732 is greater than a distance between a proximal electrode 732 and a subsequent distal electrode 731.

In some embodiments, the distance between a distal electrode 731 and a proximal electrode 732, along the length of the catheter 720, referred herein as the electrode offset 733 (FIG. 7 and FIG. 8A), is from about 0.25 mm to about 16 mm (e.g., from about 0.5 mm to about 15 mm, from about 1 mm to about 14 mm, from about 2 mm to about 13 mm, from about 3 mm to about 12 mm, or from about 4 mm to about 10 mm). In some embodiments, the electrode offset 733 is at least about 0.25 mm, 0.5 mm, 1 mm, 2 mm, 3 mm, 4 mm, 5 mm, 7 mm, 7 mm, 8 mm, 9 mm, 10 mm, 11 mm, 12 mm, 13 mm, 14 mm, or 15 mm including increments therein. In some embodiments, the electrode offset 733 is at most about 0.5 mm, 1 mm, 2 mm, 3 mm, 4 mm, 5 mm, 7 mm, 7 mm, 8 mm, 9 mm, 10 mm, 11 mm, 12 mm, 13 mm, 14 mm, 15 mm, or 16 mm, including increments therein. In some embodiments, the offset 733 is established during fabrication of the ETS system, wherein the one or more pairs of electrodes 730 are secured to the catheter 720). In some embodiments, the electrode offset is measured from the distal end of the distal electrode 731 to the proximal end of the proximal electrode 732 along the longitudinal axis of the catheter 720. In some embodiments, the electrode offset is measured from the proximal end of the distal electrode 731 to the distal end of the proximal electrode 732 along the longitudinal axis of the catheter 720). In some embodiments, the electrode offset is measured from the distal end of the distal electrode 731 to the distal end of the proximal electrode 732 along the longitudinal axis of the catheter 720. In some embodiments, the electrode offset 733 is measured from the proximal end of the distal electrode 731 to the proximal end of the proximal electrode 732 along the longitudinal axis of the catheter 720. In some embodiments, the electrode offset is measured from the center of the distal electrode 731 to the center of the proximal electrode 732 along the longitudinal axis of the catheter 720. In some embodiments, the offset 733 is measured along the length of the catheter 720, for example along the spiral shape of the catheter 720 wrapping around the ET tube 200 in FIG. 7. In some embodiments, the offset is measured around the collar shape of the catheter 720 as seen in FIGS. 8A-8D. In some embodiments, one or more electrode pairs 730) are disposed around the catheter circumference as shown in FIG. 8D. In some embodiments, the catheter has an electrode portion 735 as shown in FIG. 8C. In some embodiments, the catheter has a catheter gap 740 (FIG. 8A and FIG. 8D) that runs from the proximal to distal end of the catheter. In some embodiments, the catheter gap 740 has a width, the width measured along the circumference of a collar-shaped catheter as in FIGS. 8A-8D. In some embodiments, the catheter gap 740, in a collar-shaped configuration, has a width of from 1 mm to about 10 mm (e.g., from about 1 mm to about 9 mm, from about 1 mm to about 8 mm, from about 1 mm to about 7 mm, from about 1 mm to about 6 mm, or from about 1 mm to about 5 mm). In some embodiments, the catheter gap 740, in a collar-shaped configuration, has a width of at most 10 mm (e.g., at most about 9 mm, at most about 8 mm, at most about 7 mm, at most about 6 mm, or at most about 5 mm). In some embodiments, the catheter gap 740, in a collar-shaped configuration, has a width of at least 1 mm (e.g., at least about 2 mm, at least about 3 mm, at least about 4 mm, or at least about 5 mm).

In some embodiments, the distance from a first electrode pair to a second electrode pair, measured along the spiral shape of the catheter 760 (FIG. 7), is form about 5 mm to about 110 mm (e.g., about 5 mm, about 6 mm, about 7 mm, about 8 mm, about 9) mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, or about 110 mm).

In some embodiments, the distance from a first electrode pair to a second electrode pair, measured along the length of the ET tube 770) (FIG. 7), is form about 5 mm to about 110 mm (e.g., about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40) mm, about 41 mm, about 42 mm, about 43 mm, about 44 mm, about 45 mm, about 46 mm, about 47 mm, about 48 mm, about 49 mm, about 50) mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80) mm, about 81 mm, about 82 mm, about 83 mm, about 84 mm, about 85 mm, about 86 mm, about 87 mm, about 88 mm, about 89 mm, about 90) mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, or about 110 mm).

In some embodiments, the one or more pairs of electrodes 730 contact the trachea of a subject. In some embodiments, the one or more pairs of electrodes 730 contact the tracheal wall 015 of the subject thereby enabling phrenic nerve pacing as described herein. In some embodiments, the one or more pairs of electrodes 730 are configured to contact at least about 10%, 20%, 30%, 40%, 50%, 60%, 70%, 80%, 90%, or 95% of the inner circumference of the tracheal wall of the subject. In some embodiments, the proximal electrode 732 and the distal electrode 731 contact the entire tracheal wall of the subject at a position along the proximal-distal axis of the trachea 010. In some embodiments, at least a portion of the one or more pairs of electrodes 730 contact the tracheal wall 015 of the subject.

In some embodiments, the one or more pairs of electrodes 730 are made of the same conductive material. In some embodiments, the one or more pairs of electrodes 730 are made of different conductive materials. In some embodiments, the conductive material is a material that is a conductor of electricity, which includes, for example, but is not limited to pure metal or alloys and is commonly used in the art for making wire electrodes. In some embodiments, the conductive material is selected from silicon, platinum, iridium, polyimide, gold, ceramics, nickel, titanium, copper, iron, chromium, or an alloy or combination thereof.

### Catheter

In some embodiments, the catheter 720 of the third ETS system 700 is shaped as a spiral as shown in FIG. 7. In some embodiments, the catheter 720 of the third ETS system 700 is shaped in a collar configuration as shown in FIGS. 8A-8D.

In some embodiments, the catheter 720 has a spiral shape that twists around the ET tube 200 (FIG. 7). In some embodiments, the catheter 720 has a spiral shape that makes from about 0.5 rotations to about 20 rotations around the ET tube (e.g., about 0.5 rotations, about 1 rotation, about 1.5 rotations, about 2 rotations, about 2.5 rotations, about 3 rotations, about 3.5 rotations, about 4 rotations, about 4.5 rotations, about 5 rotations, about 6 rotations, about 7 rotations, about 8 rotations, about 9 rotations, about 10 rotations, about 11 rotations, about 12 rotations, about 13 rotations, about 14 rotations, about 15 rotations, about 16 rotations, about 17 rotations, about 18 rotations, about 19 rotations, or about 20 rotations). In some embodiments, the distance along the ET tube 200 that each rotation covers from the proximal end of the ET tube to the distal end of the ET tube is from about 10 mm to about 40 mm (e.g., from about 10 mm to about 30 mm, or from about 10 mm to about 20 mm).

In some embodiments, the length of the catheter 720, in a spiral configuration (FIG. 7) along the ET tube 200, is from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm).

In some embodiments, the length of the catheter 720, in a collar configuration (FIGS. 8A-8D) along the ET tube 200, is from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm).

In some embodiments, the width of the catheter 725 (FIG. 7), in a spiral configuration is from about 5 mm to about 15 mm (e.g., from about 6 mm to about 14 mm, from about 7 mm to about 13 mm, from about 8 mm to about 12 mm, or from about 9 mm to about 11 mm). In some embodiments, the width of the catheter 725 (FIG. 7), in a spiral configuration is at least about 5 mm (e.g., at least about 6 mm, at least about 7 mm, at least about 8 mm, at least about 9 mm, or at least about 10 mm). In some embodiments, the width of the catheter 725 (FIG. 7), in a spiral configuration is at most about 15 mm (e.g., at most about 14 mm, at most about 13 mm, at most about 12 mm, at most about 11 mm, or at most about 10 mm).

In some embodiments, the catheter in a collar configuration wraps around at least a portion of the ET tube 200 as shown in FIGS. 8A-8D. In some embodiments, the catheter in a collar configuration wraps around at least an angular portion of the ET tube 200 of an angle of at least about 180 degrees, 270 degrees, or 360 degrees.

In some embodiments, the catheter 720, in a collar configuration, wraps around at most a portion of an ET tube 200 as shown in FIGS. 8A-8D. In some embodiments, the catheter includes a prong 722 to increase the catheter's coupling strength to the ET tube 200 (FIG. 8A). In some embodiments, the catheter 720 is configured to couple to the ET tube 200 by a snap-fit or a press-fit. In some embodiments, the collar-shaped catheter (FIG. 8A), the prong 722, or both has a gap 740. In some embodiments, the collar-shaped catheter and/or the size of the gap 740 are configured such that the collar-shaped catheter 720 (FIG. 8A) securely fastens to the ET tube 200. In some embodiments, the collar-shaped catheter 720 is configured to couple to the ET tube 200 by an adhesive (FIG. 8B).

### ET Tube, ET Tube Connector and Sensor

The ETS system, in general, includes an ET tube 200. The ET tube 200 is configured to be inserted into a subject's trachea 010 (FIG. 2). In some embodiments, inserting the ET tube into a subject's trachea 010 protects the subject's airway and provides ventilation during the pacing of the phrenic nerves 020 and/or 030 of the subject. In some embodiments, the ET tube is an oral ET tube 200. In some embodiments, the ET tube is a nasal ET tube. In some embodiments, the ET tube is a tracheostomy tube. In some embodiments, the ET tube is configured to function with all embodiments and/or variations of the catheter 720, ET tube connector 300, or the one or more pairs of electrodes 730 herein described.

In some embodiments, the ET tube 200 has an outer diameter of from about 3 mm to about 20 mm (e.g., from about 3 mm to about 15 mm, from about 3 mm to about 10 mm, or from about 5 mm to about 10 mm). In some embodiments, the ET tube 200 has a lumen, the lumen having a diameter of from about 2.8 mm to about 19.8 mm (e.g., from about 2.8 mm to about 15 mm, from about 2.8 mm to about 10 mm, or from about 2.8 mm to about 5 mm).

The ET tube 200, in some embodiments, has a length from a proximal end to a distal end of from 12 cm to 40 cm (e.g., about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm).

In some embodiments, the ETS platform includes an ET tube connector 300. The ET tube connector 300, in some embodiments, bridges the proximal end of the ET tube and the distal end of the ventilator tubing circuit 410 (FIG. 2). In some embodiments, the ET tube connector 300 includes a port 340 (FIG. 3).

In some embodiments, the ET tube connector includes a sensor 310 (FIG. 2). In some embodiments, the sensor is capable of measuring one or more parameters including air flow, pressure, or temperature. In some embodiments, the control unit 500 is in electrical communication with the sensor 310.

### Fourth Endotracheal Stimulation System

In another aspect, the ETS platform 1000 provided herein includes a fourth ETS system 800 as shown in FIG. 9A and FIG. 9B. The fourth ETS system 800 may, in some embodiments, include an ET tube 200, an ET tube connector 300, a catheter 820, and one or more pairs electrodes 830. FIGS. 9A-9B show an exemplary fourth ETS system 800 that includes a catheter in a collar configuration 820 and a catheter cuff 850 disposed at the distal end of the catheter 820. In some embodiments, the catheter cuff 850 can have an unexpanded configuration, as shown in FIG. 9A, and in some embodiments, the catheter cuff 850 can have an expanded configuration as shown in FIG. 9B. In some embodiments, one or more pairs of electrodes 830 are disposed on the catheter cuff 850 as shown in FIG. 9B. In some embodiments, the catheter 820 is shown configured to wrap around the endotracheal tube 200 from the proximal end of the endotracheal tube 201 to the distal end of the endotracheal tube 202. In some embodiments, each pair of electrodes 830 contains a are radially positioned around the ET tube cuff 210 (FIG. 9B). In some embodiments, the ET tube 200 includes a pilot balloon line 215 as shown in FIG. 7.

In some embodiments, the fourth ETS system 800 is configured to engage with one or more of the systems of the ETS platform 1000 including the one or more data cables 320, the control unit 500, the ventilator 400, and the ventilator tubing 410. In some embodiments, a locking mechanism 330, similar to that shown in FIG. 3 is configured to be used with the fourth ETS system 800 to secure the data cable 320 to the endotracheal tube connector via the endotracheal tube connector port 340. In some embodiments, one or more components of the fourth ETS system 800 are configured to be disposed around an outer surface of the ET tube 200 (FIGS. 9A-9B), wherein the ET tube 200 is configured to be inserted into the trachea of a subject.

### Electrodes

In some embodiments, at least a portion of the catheter 820 includes one or more pairs of electrodes 830. In some embodiments, the pair of electrodes 830 are in electrical communication with a proximal portion of the ETS system 800. In some embodiments, the one or more pairs of electrodes 830 are in electrical communication with a proximal portion of the system 800 for connection to the control unit 500. In some embodiments, the electrical communication is wired, wireless, or both. In some embodiments, the wired electrical communication employs a locking mechanism 330 to ensure that electrical communication is maintained throughout the operation of the ETS system 800.

In some embodiments, the one or more pairs of electrodes 830 are located on an outer surface of the catheter cuff 850 (FIG. 9B). In some embodiments, the pair of electrodes 830 are bipolar. In some embodiments, the bipolar electrodes enable pacing of the phrenic nerve that runs parallel to the trachea regardless of the angular orientation of the inner catheter with respect to the trachea 010. In some embodiments, the pair of electrodes 830 are monopolar. In some embodiments, the one or more pairs of electrodes 830 are evenly distributed along a radial length around catheter cuff 850. In some embodiments, the one or more pairs of electrodes 830 are not evenly distributed along the length of the catheter cuff 850. In some embodiments, the one or more pairs of electrodes 830 are disposed at least partially around the circumference of the catheter cuff 850 (FIG. 9B).

In some embodiments, the one or more pairs of electrodes 830 have a circular shape. In some embodiments, the one or more pairs of electrodes are rectangular shaped. In some embodiments, the one or more pairs of electrodes have a circular, a rectangular, a triangular, an oval, a D-shape, a star shape, a polygonal shape, or any combination or variation thereof.

In some embodiments, the one or more pairs of electrodes 830, when shaped in a circular configuration (FIG. 7), have a diameter of from about 1 mm to 10 mm (e.g., from about 2 mm to 10 mm, from 3 mm to about 10 mm, from about 4 mm to about 10 mm, from about 5 mm to about 10 mm, or from about 6 mm to about 10 mm). In some embodiments, the one or more pairs of electrodes 830 have a diameter of at least about 1 mm, about 2 mm, about 3 mm, about 4 mm, or about 5 mm. In some embodiments, the one or more pairs of electrodes have a diameter of at most about 10 mm, about 9 mm, about 8 mm, about 7 mm, or about 6 mm.

In some embodiments, the one or more pairs of electrodes 830, when shaped in a rectangular configuration, have a length and a width wherein the length is the dimension of the electrode that extends around the circumference of the ET tube and the width is the dimension of the electrode from the proximal end of the ET tube to the distal end of the ET tube. In some embodiments, the length of an electrode of the one or more pairs of electrodes is from about 3 mm to about 20 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm).

In some embodiments, the width of an electrode of the one or more pairs of electrodes 830 is from about 1 mm to about 10 mm (e.g., from about 2 mm to about 10 mm, or from about 5 mm to about 10 mm). In some embodiments, the width of an electrode of the one or more pairs of electrodes 830 is at least about 1 mm, about 2 mm, or about 5 mm. In some embodiments, the width of an electrode of the one or more pairs of electrodes 830 is at most about 10 mm, about or about 5 mm.

In some embodiments, the distance between a pair of electrodes 830, along the circumference of the catheter cuff 850 in an expanded configuration, is referred herein as the electrode offset 833 (FIG. 9B), and is from about 1 mm to about 10 mm (e.g., from about 2 mm to about 10 mm, or from about 5 mm to about 10 mm). In some embodiments, the electrode offset 833 is at least about 1 mm, 2 mm, 3 mm, 4 mm, 5 mm, 7 mm, 7 mm, 8 mm, 9 mm, or 10 mm including increments therein. In some embodiments, the electrode offset 733 is at most about 1 mm, 2 mm, 3 mm, 4 mm, 5 mm, 7 mm, 7 mm, 8 mm, 9 mm, or 10 mm, including increments therein. In some embodiments, the offset 833 is established during fabrication of the ETS system, wherein the one or more pairs of electrodes 830 are secured to the catheter 820. In some embodiments, the electrode offset is measured from the center of one electrode to the center of a neighboring electrode along the radial axis around the catheter cuff 850.

In some embodiments, the one or more pairs of electrodes 830 contact the trachea of a subject. In some embodiments, the one or more pairs of electrodes 830 contact the tracheal wall 015 of the subject thereby enabling phrenic nerve pacing as described herein. In some embodiments, the one or more pairs of electrodes 830 are configured to contact at least about 10%, 20%, 30%, 40%, 50%, 60%, 70%, 80%, 90%, or 95% of the inner circumference of the tracheal wall of the subject. In some embodiments, at least a portion of the one or more pairs of electrodes 830 contact the tracheal wall 015 of the subject.

In some embodiments, the one or more pairs of electrodes 830 are made of the same conductive material. In some embodiments, the one or more pairs of electrodes 830 are made of different conductive materials. In some embodiments, the conductive material is a material that is a conductor of electricity, which includes, for example, but is not limited to pure metal or alloys and is commonly used in the art for making wire electrodes. In some embodiments, the conductive material is selected from silicon, platinum, iridium, polyimide, gold, ceramics, nickel, titanium, copper, iron, chromium, or an alloy or combination thereof.

### Catheter

In some embodiments, the catheter 820 of the fourth ETS system 800 is shaped as a collar having a catheter cuff 850 at a distal end of the catheter as shown in FIGS. 9A-9B. In some embodiments, the catheter 820 is configured and sized to be translated along an ET tube 200 to the site of endotracheal stimulation. In some embodiments, the catheter 820 is configured to be translated along the length of the ET tube 200 having the catheter cuff 850 in an unexpanded configuration (FIG. 9A). In some embodiments, the catheter 820 is configured to position the catheter cuff 850 over the ET cuff 210 as shown in FIG. 9B. In some embodiments, the inflation state of the ET cuff 210 determines the configuration from unexpanded to expanded of the catheter cuff 850. In some embodiments, the catheter cuff 850 is configured to slide over the ET cuff 210 and is configured to be expanded by either inflation of the ET cuff 210 or by inflation of the catheter cuff 850.

In some embodiments, the catheter cuff 850 has one or more electrodes positioned radially around the ET tube 200. In some embodiments, each electrode of the catheter cuff is supported by an expandable scaffolding material (e.g., a wire, a net, a mesh or any variants and/or combinations thereof). In some embodiments, the catheter cuff 850 has from 1 to 20 electrodes (e.g., from 1 to 18, from 1 to 16, from 1 to 14 electrodes, from 1 to 12 electrodes, from 1 to 10 electrodes, from 1 to 8 electrodes, or from 1 to 6 electrodes). In some embodiments, the catheter cuff has at least 1, 2, 3, 4, 5, 6, 7, 8, 9, 10, 11, 12, 13, 14, or 15 electrodes. In some embodiments, the catheter cuff has at most 20, 19, 18, 17, 16, 15, 14, 13, 12, 11, 10, 9, 8, 7, 6, 5, 4, 3, or 2 electrodes.

In some embodiments, the length of the catheter 820, along the ET tube 200, is from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm).

In some embodiments, the catheter wraps around at least a portion of the ET tube 200 as shown in FIGS. 9A-9B. In some embodiments, the catheter wraps around at least an angular portion of the ET tube 200 of an angle of at least about 180 degrees, 270 degrees, or 360 degrees.

In some embodiments, the catheter includes a prong to increase the catheter's coupling strength to the ET tube 200 (FIG. 8A). In some embodiments, the catheter 820 is configured to couple to the ET tube 200 by a snap-fit or a press-fit.

### ET Tube, ET Tube Connector and Sensor

The ETS system, in general, includes an ET tube 200. The ET tube 200 is configured to be inserted into a subject's trachea 010 (FIG. 2). In some embodiments, inserting the ET tube into a subject's trachea 010 protects the subject's airway and provides ventilation during the pacing of the phrenic nerves 020 and/or 030 of the subject. In some embodiments, the ET tube is an oral ET tube 200. In some embodiments, the ET tube is a nasal ET tube. In some embodiments, the ET tube is a tracheostomy tube. In some embodiments, the ET tube is configured to function with all embodiments and/or variations of the catheter 820, ET tube connector 300, or the one or more pairs of electrodes 830 herein described.

In some embodiments, the ET tube 200 has an outer diameter of from about 3 mm to about 20 mm (e.g., from about 3 mm to about 18 mm, from about 4 mm to about 16 mm, from about 5 mm to about 14 mm, from about 6 mm to about 13 mm, from about 7 mm to about 12 mm, or from about 8 mm to about 11 mm). In some embodiments, the ET tube 200 has an outer diameter of at most 20 mm (e.g., at most 15, at most 10 mm, at most 5 mm). In some embodiments, the ET tube 200 has a lumen, the lumen having a diameter of from about 2.8 mm to about 19.8 mm (e.g., from about 2.8 mm to about 15 mm, from about 2.8 mm to about 10 mm, or from about 2.8 mm to about 5 mm).

The ET tube 200, in some embodiments, has a length from a proximal end to a distal end of from 12 cm to 40 cm (e.g., about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm).

In some embodiments, the ETS platform includes an ET tube connector 300. The ET tube connector 300, in some embodiments, bridges the proximal end of the ET tube and the distal end of the ventilator tubing circuit 410 (FIG. 2). In some embodiments, the ET tube connector 300 includes a port 340 (FIG. 3).

In some embodiments, the ET tube connector includes a sensor 310 (FIG. 2). In some embodiments, the sensor is capable of measuring one or more parameters including air flow, pressure, or temperature. In some embodiments, the control unit 500 is in electrical communication with the sensor 310.

### Methods of Pacing a Subject

In another aspect, the invention provides a method of pacing a subject, the method including inserting one or more components of an ETS system, as herein described, into an ET tube 200 of a subject; contacting the wall of the trachea with the pair of electrodes by advancing a catheter out of the guide sheath to convert the first ETS system from the constrained configuration to the unconstrained configuration; and providing electrical power to the pair of electrodes in a pattern to pace the phrenic nerve of the subject. In some embodiments, the method further includes flexing the catheter, deforming the catheter, or both.

In some embodiments, inserting an ETS system, as herein described, into the trachea of a subject includes inserting an ET tube into the trachea of the subject and inserting the ETS system into the ET tube. In some embodiments, inserting the ETS system herein into the trachea of a subject includes inserting an ET tube into the trachea of the subject and inserting a catheter and a guide sheath together into the ET tube. In some embodiments, inserting the ETS system herein into the trachea of a subject includes inserting the ET tube into the trachea of the subject and inserting the guide sheath into the ET tube and inserting the catheter into the guide sheath.

In some embodiments, the method further includes intubating the subject with an ET tube, connecting the ET tube to a mechanical ventilator. In some embodiments, the method further includes flexing and/or shaping the catheter to a specific shape. In some embodiments, the method further includes confirming, with the pair of electrodes and/or another sensing device, that the catheter has been properly placed (e.g., near the carina)

In some embodiments, the ETS system does not include the catheter, wherein the pairs of electrodes are advanced out of the guide sheath. In some embodiments, the ETS system does not include the catheter, wherein the pairs of electrodes are flexed, deformed, or both. In some embodiments, the ETS system does not include the catheter, wherein the pairs of electrodes and the guide sheath together into the ET tube.

In another aspect, the invention provides a method of pacing a subject, the method including inserting the second endotracheal system, as herein described, into an ET tube of a subject; contacting a wall of the trachea with the pair of electrodes by advancing a catheter out of a guide sheath to convert the ETS system from the constrained configuration to the unconstrained configuration; and providing electrical power to the pair of electrodes in a pattern to pace the phrenic nerve of the subject. In some embodiments, the method further includes flexing the catheter, deforming the catheter, or both.

In some embodiments, the ETS system does not include a catheter, wherein the pairs of electrodes are advanced out of the guide sheath. In some embodiments, the ETS system does not include a catheter, wherein the pairs of electrodes are flexed, deformed, or both. In some embodiments, the ETS system does not include a catheter, wherein the pairs of electrodes and the guide sheath together into the ET tube.

In another aspect the invention provides a method of pacing a subject, the method including inserting the third endotracheal system herein into the trachea of a subject; contacting a wall of the tracheal with the pair of electrodes; and providing electrical power to the pair of electrodes in a pattern to pace the phrenic nerve of the subject. In some embodiments, the method further includes placing the third endotracheal system over an ET tube.

In another aspect the invention provides a method of pacing a subject, the method including inserting the fourth endotracheal system herein into the trachea of a subject; contacting a wall of the tracheal with the pair of electrodes; and providing electrical power to the pair of electrodes in a pattern to pace the phrenic nerve of the subject. In some embodiments, the method further includes placing the fourth endotracheal system over an ET tube.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “one or more”, “at least one”, “more than one”, and the like are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

The term “endotracheal stimulation platform,” as used herein, refers to one or more ETS systems and devices or systems functionally connected therewith. For example, an “endotracheal stimulation platform” may include any one of more ETS systems as herein described and one or more control units and/or one or more ventilators.

As used herein, the term “distal” refers to the end farthest away from the medical professional when introducing a device into a patient, while the term “proximal” refers to the end closest to the medical professional when introducing a device into a patient.

The term “longitudinal axis”, as used herein, refers to the exact or approximate central axis, with respect to a device or component thereof, along its greater dimension, i.e., along its length, from its distal end to its proximal end, and vice versa, and is not intended to be limited to imply a straight line, wherein, for example, a catheter includes a spiral shape as described herein, it is intended that “longitudinal axis” as used herein follows such spiral.

As used herein, the term “expandable” refers to the ability to increase in diameter from a “collapsed” or “deflated” configuration to an “expanded” or “inflated” configuration. As used herein, “diameter” refers to the distance of a straight line extending between two points and does not necessarily indicate a particular shape.

The terms “constrained,” “undeployed,” “unexpanded,” or variants thereof, as used herein, may be used interchangeably and intended to refer to a collapsed or compressed configuration having a reduced physical footprint compared to an “unconstrained” configuration.

The terms “unconstrained,” “deployed,” “expanded,” or variants thereof, as used herein, may be used interchangeably and intended to refer to an enlarged and/or unfurled configuration having an increased physical footprint compared to an “constrained” configuration.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. An endotracheal stimulation system comprising:
(a) a catheter wherein at least a distal portion of the catheter comprises a pair of electrodes comprising a proximal looped electrode and a distal looped electrode, wherein the proximal looped electrode and the distal looped electrode are in electrical communication with a proximal portion of the system for connection to a control unit; and
(b) a guide sheath having a lumen surrounding at least a portion of the catheter and sized to fit within an endotracheal tube, wherein the catheter is axially translatable with respect to the endotracheal tube;
wherein the endotracheal stimulation system has:
(c) a constrained configuration when at least a portion of the proximal looped electrode and the distal looped electrode are within the guide sheath; and
(d) an unconstrained configuration when at least a portion of the proximal looped electrode and the distal looped electrode extends out of the guide sheath such that the pair of electrodes contact the trachea of a subject.

2. The system of claim 1, wherein the proximal looped electrode and the distal looped electrode are bipolar.

3. The system of claim 1, wherein the proximal looped electrode and the distal looped electrode are monopolar.

4. The system of claim 1, 2, or 3, wherein the proximal looped electrode is coated with a proximal insulation, wherein the distal looped electrode is coated with a distal insulation, or both.

5. The system of any one of claims 1-4, wherein the proximal looped electrode and the distal looped electrode are electrically isolated in the constrained configuration, the unconstrained configuration, or both.

6. The system of any one of claims 1-5, wherein a proximal point of the proximal looped electrode is proximal to a proximal point of the distal looped electrode by an electrode offset in the constrained configuration, the unconstrained configuration, or both.

7. The system of claim 6, wherein the electrode offset is measured as a distance between a distal point of the proximal looped electrode and a distal point of the distal looped electrode.

8. The system of claim 7, wherein the electrode offset is about 0.25 cm to about 40 cm.

9. An endotracheal stimulation system comprising:
(a) a catheter wherein at least a distal portion of the catheter comprises a pair of electrodes, wherein the pair of electrodes are in electrical communication with a proximal portion of the system for connection to a control unit, and wherein the distal portion of the catheter is biased to have a shape that is non-straight; and
(b) a guide sheath having a lumen surrounding at least a portion of the catheter and sized to fit within an endotracheal tube, wherein the catheter is axially translatable with respect to the guide sheath;
wherein the endotracheal stimulation system has:
(c) a constrained configuration when the distal portion of the catheter is within the guide sheath, and wherein an axis of the catheter and an axis of the guide sheath are axially aligned; and
(d) an unconstrained configuration when the distal portion of the catheter extends out of the guide sheath, wherein at least a portion of the axis of the catheter and the axis of the guide sheath are askew, such that the pair of electrodes contact a trachea of a subject.

10. The system of claim 9, wherein the pair of electrodes are bipolar.

11. The system of claim 9, wherein the pair of electrodes are monopolar.

12. The system of claim 9, 10, or 11, wherein the distal portion of the catheter comprises two or more pairs of electrodes.

13. The system of claim 12, wherein the two or more pairs of electrodes are evenly distributed along the length of the catheter.

14. The system of claim 12 or 13, wherein each of the two or more pairs of electrodes are separated by an offset distance of about 0.25 cm to about 16 cm.

15. The system of any one of claims 9-14, wherein the catheter further comprises a lumen.

16. The system of any one of claims 9-15, wherein the catheter is flexible, pliable, or both.

17. The system of any one of claims 9-16, wherein the catheter has a non-straight shape in the unconstrained configuration with a diameter of about 1 mm to about 35 mm.

18. The system of claim 17, wherein the catheter has a non-straight shape in the unconstrained configuration with a minimum, average, or maximum arc angle of about 90 degrees to about 360 degrees.

19. An endotracheal stimulation system comprising: a catheter at the distal end of the system configured to wrap around at least a portion of an endotracheal tube, wherein the catheter comprises a pair of electrodes on an outer catheter surface, and wherein the pair of electrodes are in electrical communication with a proximal portion of the system for connection to a control unit.

20. The system of claim 19, wherein the pair of electrodes are bipolar.

21. The system of claim 19, wherein the pair of electrodes are monopolar.

22. The system of claim 19, 20, or 21, wherein the catheter comprises two or more pairs of electrodes.

23. The system of any one of claims 19-22, wherein the two or more pairs of electrodes are evenly distributed along the length of the catheter.

24. A method of pacing a subject, the method comprising:
(a) inserting the system of any one of claims 1-8 into a tracheal tube of a subject;
(b) contacting a wall of the trachea with the pair of electrodes by advancing the catheter out of the guide sheath to convert the endotracheal stimulation system from the constrained configuration to the unconstrained configuration; and
(c) providing electrical power to the pair of electrodes in a pattern to pace the phrenic nerve of the subject.

25. The method of claim 24, further comprising flexing the catheter, deforming the catheter, or both.

26. A method of pacing a subject, the method comprising:
(a) inserting the system of any one of claims 9-18 into a tracheal tube of a subject;
(b) contacting a wall of the trachea with the pair of electrodes by advancing the catheter out of the guide sheath to convert the endotracheal stimulation system from the constrained configuration to the unconstrained configuration; and
(c) providing electrical power to the pair of electrodes in a pattern to pace the phrenic nerve of the subject.

27. The method of claim 26, further comprising flexing the catheter, deforming the catheter, or both.

28. A method of pacing a subject, the method comprising:
(a) inserting the system of any one of claims 19-25 into the trachea of a subject;
(b) contacting a wall of the trachea with the pair of electrodes; and
(c) providing electrical power to the pair of electrodes in a pattern to pace the phrenic nerve of the subject.
